(19) United States
(12) Patent Application Publication
(10) Pub. No.: US 2025/0145677 A1
(43) Pub. Date: May 8, 2025

(54) ENGINEERED SEMAPHORINS AND USES THEREOF

(71) Applicant: THE CHIDLREN'S MEDICAL CENTER CORPORATION, Boston, MA (US)

(72) Inventors: David M. BRISCOE, Sharon, MA (US); Diane BIELENBERG, Andover, MA (US); Johannes WEDEL, Jamaica Plain, MA (US)

(73) Assignee: THE CHILDREN'S MEDICAL CENTER CORPORATION, Boston, MA (US)

(21) Appl. No.: 18/788,401

(22) Filed: Jul. 30, 2024

Related U.S. Application Data
(63) Continuation of application No. 18/119,920, filed on Mar. 10, 2023, now abandoned.
Application No. 18/119,920 is a continuation of application No. 17/271,380, filed on Feb. 25, 2021, now abandoned.
(60) Provisional application No. 62/723,756, filed on Aug. 28, 2018.

(51) Int. Cl. C07K 14/47; A61K 38/00; A61P 37/06

(52) U.S. Cl. CPC C07K 14/4703; A61P 37/06; A61K 38/00

(57) ABSTRACT

Provided herein are methods and compositions comprising or using mutant semaphorin polypeptides.

### CROSS-REFERENCE TO RELATED APPLICATIONS

This application is a continuation under 35 U.S.C. § 120 of co-pending U.S. application Ser. No. 18/119,920 filed Mar. 10, 2023, which is a continuation under 35 U.S.C. § 120 of U.S. application Ser. No. 17/271,380 filed Feb. 25, 2021 now abandoned, which is a 371 National Phase Entry of International Patent Application No. PCT/US2019/048267, filed on Aug. 27, 2019, which claims benefit under 35 U.S.C. § 119 of U.S. Provisional Application No. 62/723,756 filed Aug. 28, 2018, the contents of each of which are incorporated herein by reference in their entireties.

### GOVERNMENT SUPPORT

This invention was made with government support under Grant No. AI092305, awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on April 3, 2023, is named 701039-089190USC1_SL.xml and is 33,084 bytes in size.

### FIELD OF THE INVENTION

The field of the invention relates to immunomodulation and the treatment of cancer.

### BACKGROUND

The class 3 family of semaphorins (Sema3A-G) bind to Neuropilin and Plexin family proteins and elicit regulatory signals that inhibit cellular migration and proliferation. Specifically, the binding of SEMA3A to NRP-1 and SEMA3F to NRP-2 elicits inhibitory signals in neuronal cells and in vascular endothelial cells.

### SUMMARY

The methods, compositions and treatments described herein are based, in part, on the discovery that semaphorins have immunomodulatory activities and that, for example, semaphorin agonists inhibit or prevent transplant rejection, among other therapeutic approaches. The methods compositions, and treatments described herein are also based, in part, on the discovery that targeted mutation of the semaphorin polypeptides can increase their in vivo half-life. Thus, the methods, compositions and treatments described herein are based, in part, on engineered semaphorins that have an extended half-life in vivo compared to a wild-type semaphorin, for example, the semaphorin from which it is derived. This extended half-life in vivo permits the improved use of compositions comprising such engineered semaphorins in any setting in which a semaphorin agonist is beneficial. Non-limiting examples include use in inflammatory disease, including chronic inflammatory disease (e.g., arthritis, inflammatory bowel disease, psoriasis etc.), and cancer (including, but not limited to cancers that express neuropilin-2).

Accordingly, provided herein in one aspect, is a mutant semaphorin (SEMA) polypeptide in which the proproteinase cleavage motif R-X-X-R (e.g., RRSRR (SEQ ID NO: 8)) is inactivated by mutation. Preferred proproteinase cleavage motifs for mutation include R-X-Lys/Arg-R. In some embodiments, the proproteinase cleavage motif is RRFRR (SEQ ID NO: 10), RRTRR (SEQ ID NO: 11), RFRR (SEQ ID NO: 12), RTRR (SEQ ID NO: 13), RSRR (SEQ ID NO: 14), RRSRR (SEQ ID NO: 8), etc. In one embodiment, the proproteinase cleavage site is recognized by furin, a ubiquitous subtilisin-like proprotein convertase.

In one embodiment of this aspect and all other aspects provided herein, the wild-type SEMA polypeptide binds to neuropilin 2, and the mutant SEMA polypeptide retains the ability to bind to neuropilin 2.

In another embodiment of this aspect and all other aspects provided herein, the SEMA polypeptide is a human SEMA polypeptide.

In another embodiment of this aspect and all other aspects provided herein, the SEMA polypeptide is selected from a SEMA 3A polypeptide, SEMA 3C polypeptide, a SEMA 3F polypeptide and a SEMA 3G polypeptide.

In another embodiment of this aspect and all other aspects provided herein, the proproteinase cleavage motif RRSRR (SEQ ID NO: 8) comprises amino acids (i) 582-586 of SEQ ID NO: 1, (ii) 583-586 of SEQ ID NO: 1, (iii) 550-555 of SEQ ID NO: 5, (iv) 551-555 of SEQ ID NO: 5, (v) 548-552 of SEQ ID NO: 6, (vi) 549-552 of SEQ ID NO: 6, (vii) 557-561 of SEQ ID NO: 7, or (viii) 558-561 of SEQ ID NO: 7. In one embodiment of this aspect and all other aspects described herein, the non-mutant semaphorin polypeptide comprises the sequence at UniProtKB—Q13275 (SEM3F_HUMAN; gene ID: 6405, location 3p21.31).

In another embodiment of this aspect and all other aspects provided herein, the proproteinase cleavage motif RRSRR (SEQ ID NO: 8) is inactivated by mutating the second and fourth arginines in the motif.

In another embodiment of this aspect and all other aspects provided herein, the proproteinase cleavage motif RRSRR (SEQ ID NO: 8) is inactivated by mutating arginine 583 and arginine 586 of SEQ ID NO: 1 to alanine.

Another aspect provided herein relates to a nucleic acid molecule encoding any one of the mutant SEMA polypeptides as described herein.

In one embodiment of this aspect and all other aspects provided herein, the nucleic acid molecule is a cDNA or a modified RNA.

Another aspect provided herein relates to a vector comprising a nucleic acid molecule encoding any one of the mutant SEMA polypeptides described herein.

In one embodiment of this aspect and all other aspects provided herein, the vector is a viral vector.

In another embodiment of this aspect and all other aspects provided herein, the viral vector is an adenoviral vector or an adeno-associated viral (AAV) vector.

Also provided herein, in another aspect is a cell comprising a nucleic acid encoding any one of the mutant SEMA polypeptides, or a vector comprising a nucleic acid encoding any one of the mutant SEMA polypeptides.

Another aspect provided herein relates to a pharmaceutical composition comprising the mutant SEMA polypeptide as described herein, the nucleic acid encoding such mutant SEMA polypeptides, the vector encoding such mutant SEMA polypeptides, or a cell comprising a mutant SEMA polypeptide, nucleic acid or vector thereof.

Also provided herein is a method of inhibiting transplant or allograft rejection in a subject in need thereof, comprising administering to the subject a therapeutically effective amount of a pharmaceutical composition as described herein.

Another aspect provided herein relates to a method of inhibiting transplant or allograft rejection comprising contacting transplant tissue with a pharmaceutical composition as described herein.

Also provided herein, in another aspect, is a method of suppressing the immune system in a subject in need thereof comprising administering to the subject an amount of a pharmaceutical composition (as described herein) effective to suppress the immune system of the subject.

An alternative aspect described herein relates to a method of treating an inflammatory condition in a subject in need thereof comprising administering to the subject a pharmaceutical composition as described herein.

In one embodiment of this aspect and all other aspects provided herein, the inflammatory condition is an autoimmune disease.

In another embodiment of this aspect and all other aspects provided herein, the transplant tissue is contacted in vivo prior to removal from a tissue donor.

In another embodiment of this aspect and all other aspects provided herein, the transplant tissue is contacted ex vivo or in vitro.

Another aspect provided herein relates to a multispecific agent comprising a semaphorin polypeptide that binds to neuropilin 2, and an agent that binds an immunomodulator polypeptide.

In one embodiment of this aspect and all other aspects provided herein, the semaphorin polypeptide comprises a semaphorin family immunoglobulin domain.

In another embodiment of this aspect and all other aspects provided herein, the semaphorin family immunoglobulin domain is a SEMA3F immunoglobulin domain.

In another embodiment of this aspect and all other aspects provided herein, the semaphorin polypeptide is selected from semaphorin 3F, semaphorin 3G, semaphorin 3A, and semaphorin 3C.

In another embodiment of this aspect and all other aspects provided herein, the immunomodulator polypeptide is an immune checkpoint polypeptide.

In another embodiment of this aspect and all other aspects provided herein, the immune checkpoint polypeptide is selected from PD-L1, TIM-1, TIM-3, PD-1, CTLA4, TIGIT, LAG3, VISTA, 4-1BBL, B7-H3 and B7-DC.

In another embodiment of this aspect and all other aspects provided herein, the agent that binds an immunomodulator polypeptide inhibits the immune checkpoint polypeptide.

In another embodiment of this aspect and all other aspects provided herein, the semaphorin polypeptide has a mutation that inactivates the proproteinase cleavage site RRSRR (SEQ ID NO: 8).

Also provided herein, in another aspect, is a composition comprising a first semaphorin polypeptide and a second semaphorin polypeptide, joined by a linker.

In another embodiment of this aspect and all other aspects provided herein, the first semaphorin polypeptide and the second semaphorin polypeptide are the same.

Another aspect provided herein relates to a method of treating cancer, the method comprising administering to a subject in need thereof a composition comprising a mutant SEMA polypeptide as described herein.

In one embodiment, the method further comprises administering an immune checkpoint inhibitor. In another embodiment, the immune checkpoint inhibitor inhibits a checkpoint molecule selected from the group consisting of PD-L1, TIM-1, TIM-3, PD-1, CTLA4, TIGIT, LAG3, VISTA, 4-1BBL, B7-H3 and B7-DC.

In another embodiment, the method further comprises administering an inhibitor of neuropilin-2.

In another embodiment, the method further comprises administering a chemotherapeutic or anti-cancer agent, or radiation treatment.

In another embodiment, the cancer expresses neuropilin-2.

Another aspect provided herein relates to a method of treating cancer, the method comprising administering to a subject in need thereof a pharmaceutical composition as described herein.

In one embodiment, the method further comprises administering an immune checkpoint inhibitor. In another embodiment, the immune checkpoint inhibitor inhibits a checkpoint molecule selected from the group consisting of PD-L1, TIM-1, TIM-3, PD-1, CTLA4, TIGIT, LAG3, VISTA, 4-1BBL, B7-H3 and B7-DC.

In another embodiment, the method further comprises administering an inhibitor of neuropilin-2.

In another embodiment, the method further comprises administering a chemotherapeutic or anti-cancer agent, or radiation treatment.

In another embodiment, the cancer expresses neuropilin-2.

Another aspect provided herein relates to a method of treating cancer, the method comprising administering to a subject in need thereof a multispecific agent as described herein.

In one embodiment, the method further comprises administering an immune checkpoint inhibitor. In another embodiment, the immune checkpoint inhibitor inhibits a checkpoint molecule selected from the group consisting of PD-L1, TIM-1, TIM-3, PD-1, CTLA4, TIGIT, LAG3, VISTA, 4-1BBL, B7-H3 and B7-DC.

In another embodiment, the method further comprises administering an inhibitor of neuropilin-2.

In another embodiment, the method further comprises administering a chemotherapeutic or anti-cancer agent, or radiation treatment.

In another embodiment, the cancer expresses neuropilin-2.

In one embodiment, the cancer expresses neuropilin-2.

Another aspect provided herein relates to a method of inhibiting metastasis of a cancer, the method comprising administering to a subject in need thereof a composition comprising a mutant SEMA polypeptide as described herein. In one embodiment, the cancer expresses neuropilin-2.

Also provided herein, in another aspect is a method of inhibiting metastasis of a cancer, the method comprising administering to a subject in need thereof a pharmaceutical composition as described herein. In one embodiment, the cancer expresses neuropilin-2.

Another aspect provided herein relates to a method of inhibiting metastasis of a cancer, the method comprising administering to a subject in need thereof a multispecific agent as described herein.

In one embodiment, the cancer expresses neuropilin-2.

In one aspect, described herein is a method of suppressing the immune system in a subject, the method comprising administering a mutated SEMA polypeptide as described herein to a subject in need thereof. In some embodiments, suppression of the immune system can comprise treating an inflammatory condition. In some embodiments, suppression of the immune system can comprise suppressing graft rejection (e.g., allograft rejection) or the like. In one aspect, described herein is a method of inhibiting Akt/mTOR signaling in a cell, the method comprising contacting the cell with a mutant SEMA polypeptide as described herein. In one aspect, described herein is a method of inhibiting Akt/mTOR signaling in a subject, the method comprising administering a mutant semaphorin as described herein to a subject in need thereof.

### BRIEF DESCRIPTION OF THE DRAWINGS

FIG. 1 shows graphs of cell number versus concentration of wild-type (left panel; “SEMA3F”) and mutant (right panel; “SEMA3F furin uncleavable”) Sema3F polypeptides when introduced to cultured human umbilical vein endothelial cells (HUVEC). Results from cultures in the presence (+heparin) and absence (−heparin) of heparin are shown for each Sema3F polypeptide.

FIG. 2 shows the staining of f-actin filaments in HUVECs cultured with wild-type (top panels; “SEMA3F”) and mutant, non-cleavable (bottom panels; “Nc SEMA3F) Sema3F polypeptides.

FIG. 3 shows results of a chemorepulsion assay on cultured endothelial cells using cells expressing wild-type (top panels; “WT3F (A3) and mutant non-cleavable (bottom panels; “Δ3F (13)”).

FIG. 4 shows generation of SEMA Δ3F. The recognition sequence of Sema3F-Fu1 is Arg-Arg-Ser-Arg-Arg (SEQ ID NO: 8). The mutation of the recognition sequence is Arg-Ala-Ser-Arg-Ala (SEQ ID NO: 9).

### DETAILED DESCRIPTION

Provided herein are methods and compositions for immunomodulation based on engineered semaphorin(s) comprising an extended in vivo length of activity (i.e., a longer half-life) relative to the wild-type semaphorin(s) from which they were designed. Immunomodulation using such compositions can be used in the treatment of inflammatory diseases, including chronic inflammatory diseases, and cancer.

### Inflammatory Diseases and Disorders

As used herein, “inflammation” refers to the complex biological response to harmful stimuli, such as pathogens, damaged cells, or irritants. Inflammation is a protective attempt by the organism to remove the injurious stimuli as well as initiate the healing process for the tissue. Accordingly, the term “inflammation” includes any cellular process that leads to the production of pro-inflammatory cytokines, inflammation mediators and/or the related downstream cellular events resulting from the actions of the cytokines thus produced, for example, fever, fluid accumulation, swelling, trafficking or accumulation of immune or inflammatory cell types, abscess formation, and cell death. Pro-inflammatory cytokines and inflammation mediators include, but are not limited to, IL-1-alpha, IL-1-beta, IL-6, IL-8, IL-11, IL-12, IL-17, IL-18, TNF-alpha, leukocyte inhibitory factor (LIF), IFN-gamma, Oncostatin M (OSM), ciliary neurotrophic factor (CNTF), TGF-beta, granulocyte-macrophage colony stimulating factor (GM-CSF), and chemokines that chemoattract inflammatory cells. Inflammation can include both acute responses (i.e., responses in which the inflammatory processes are active) and chronic responses (i.e., responses marked by slow progression and formation of new connective tissue). Acute and chronic inflammation can be distinguished by the cell types involved. Acute inflammation often involves polymorphonuclear neutrophils; whereas chronic inflammation is normally characterized by a lymphohistiocytic and/or granulomatous response.

An inflammatory condition is any disease state characterized by inflammatory tissues (for example, infiltrates of leukocytes such as lymphocytes, neutrophils, macrophages, eosinophils, mast cells, basophils and dendritic cells) or inflammatory processes which provoke or contribute to the abnormal clinical and histological characteristics of the disease state. Inflammatory conditions include, but are not limited to, inflammatory conditions of the skin, inflammatory conditions of the lung, inflammatory conditions of the joints, inflammatory conditions of the gut, inflammatory conditions of the eye, inflammatory conditions of the endocrine system, inflammatory conditions of the cardiovascular system, inflammatory conditions of the kidneys, inflammatory conditions of the liver, inflammatory conditions of the central nervous system, or sepsis-associated conditions. In some embodiments, the inflammatory condition is associated with wound healing. In some embodiments, the inflammation to be treated according to the methods described herein can be skin inflammation; inflammation caused by substance abuse or drug addiction; inflammation associated with infection; inflammation of the cornea; inflammation of the retina; inflammation of the spinal cord; inflammation associated with organ regeneration; and pulmonary inflammation.

In some embodiments, an inflammatory condition can be an autoimmune disease. Non-limiting examples of autoimmune diseases can include: Type 1 diabetes; systemic lupus erythematosus; rheumatoid arthritis; psoriasis; inflammatory bowel disease; Crohn's disease; and autoimmune thyroiditis. Autoimmune diseases are well known in the art; for example, see “Autoimmune Diseases Research Plan” Autoimmune Disease Coordinating Committee. NIH Publication No. 03-510, December 2002; which is incorporated by reference herein in its entirety.

In some embodiments, a subject in need of treatment for inflammation, wound healing, or pain management can be a subject having, or diagnosed as having temporomandibular joint disorders; COPD; smoke-induced lung injury; renal dialysis associated disorders; spinal cord injury; graft vs. host disease; bone marrow transplant or complications thereof; infection; trauma; pain; incisions; surgical incisions; a chronic pain disorder; a chronic bone disorder; mastitis; and joint disease. In some embodiments, trauma can include battle-related injuries or tissue damage occurring during a surgery. Smoke-induced lung injury can result, for example, from exposure to tobacco smoke, environmental pollutants (e.g. smog or forest fires), or industrial exposure. By way of non-limiting example, inflammatory conditions can be inflammatory conditions of the skin, such as Sweet's syndrome, pyoderma gangrenosum, subcorneal pustular dermatosis, erythema elevatum diutinum, Behcet's disease or acute generalized exanthematous pustulosis, a bullous disorder, psoriasis, a condition resulting in pustular lesions, acne, acne vulgaris, dermatitis (e.g. contact dermatitis, atopic dermatitis, seborrheic dermatitis, eczematous dermatitides, eczema craquelee, photoallergic dermatitis, phototoxic dermatitis, phytophotodermatitis, radiation dermatitis, stasis dermatitis or allergic contact dermatitis), eczema, ulcers and erosions resulting from trauma, burns, ischemia of the skin or mucous membranes, several forms of ichthyoses, epidermolysis bullosae, hypertrophic scars, keloids, cutaneous changes of intrinsic aging, photoaging, frictional blistering caused by mechanical shearing of the skin, cutaneous atrophy resulting from the topical use of corticosteroids, and inflammation of mucous membranes (e.g., cheilitis, chapped lips, nasal irritation, mucositis and vulvovaginitis).

By way of non-limiting example, inflammatory conditions can be inflammatory conditions of the lung, such as asthma, bronchitis, chronic bronchitis, bronchiolitis, pneumonia, sinusitis, emphysema, adult respiratory distress syndrome, pulmonary inflammation, pulmonary fibrosis, and cystic fibrosis (which may additionally or alternatively involve the gastro-intestinal tract or other tissue(s)). By way of non-limiting example, inflammatory conditions can be inflammatory conditions of the joints, such as rheumatoid arthritis, rheumatoid spondylitis, juvenile rheumatoid arthritis, osteoarthritis, gouty arthritis, infectious arthritis, psoriatic arthritis, and other arthritic conditions. By way of non-limiting example, inflammatory conditions can be inflammatory conditions of the gut or bowel, such as inflammatory bowel disease, Crohn's disease, ulcerative colitis and distal proctitis. By way of non-limiting example, inflammatory conditions can be inflammatory conditions of the eye, such as dry eye syndrome, uveitis (including iritis), conjunctivitis, scleritis, and keratoconjunctivitis sicca. By way of non-limiting example, inflammatory conditions can be inflammatory conditions of the endocrine system, such as autoimmune thyroiditis (Hashimoto's disease), Graves' disease, Type I diabetes, and acute and chronic inflammation of the adrenal cortex. By way of non-limiting example, inflammatory conditions can be inflammatory conditions of the cardiovascular system, such as coronary infarct damage, peripheral vascular disease, myocarditis, vasculitis, revascularization of stenosis, artherosclerosis, and vascular disease associated with Type II diabetes. By way of non-limiting example, inflammatory conditions can be inflammatory conditions of the kidneys, such as glomerulonephritis, interstitial nephritis, lupus nephritis, and nephritis secondary to Wegener's disease, acute renal failure secondary to acute nephritis, post-obstructive syndrome and tubular ischemia. By way of non-limiting example, inflammatory conditions can be inflammatory conditions of the liver, such as hepatitis (arising from viral infection, autoimmune responses, drug treatments, toxins, environmental agents, or as a secondary consequence of a primary disorder), biliary atresia, primary biliary cirrhosis and primary sclerosing cholangitis. By way of non-limiting example, inflammatory conditions can be inflammatory conditions of the central nervous system, such as multiple sclerosis and neurodegenerative diseases such as Alzheimer's disease or dementia associated with HIV infection. By way of non-limiting example, inflammatory conditions can be inflammatory conditions of the central nervous system, such as MS; all types of encephalitis and meningitis; acute disseminated encephalomyelitis; acute transverse myelitis; neuromyelitis optica; focal demyelinating syndromes (e.g., Balo's concentric sclerosis and Marburg variant of MS); progressive multifocal leukoencephalopathy; subacute sclerosing panencephalitis; acute haemorrhagic leucoencephalitis (Hurst's disease); human T-lymphotropic virus type-1associated myelopathy/tropical spactic paraparesis; Devic's disease; human immunodeficiency virus encephalopathy; human immunodeficiency virus vacuolar myelopathy; peripheral neuropathies; Guillanne-Barre Syndrome and other immune mediated neuropathies; and myasthenia gravis. By way of non-limiting example, inflammatory conditions can be sepsis-associated conditions, such as systemic inflammatory response syndrome (SIRS), septic shock or multiple organ dysfunction syndrome (MODS). Further non-limiting examples of inflammatory conditions include, endotoxin shock, periodontal disease, polychondritis; periarticular disorders; pancreatitis; system lupus erythematosus; Sjogren's syndrome; vasculitis sarcoidosis amyloidosis; allergies; anaphylaxis; systemic mastocytosis; pelvic inflammatory disease; multiple sclerosis; multiple sclerosis (MS); celiac disease, Guillain-Barre syndrome, sclerosing cholangitis, autoimmune hepatitis, Raynaud's phenomenon, Goodpasture's syndrome, Wegener's granulomatosis, polymyalgia rheumatica, temporal arteritis/giant cell arteritis, chronic fatigue syndrome CFS), autoimmune Addison's Disease, ankylosing spondylitis, Acute disseminated encephalomyelitis, antiphospholipid antibody syndrome, aplastic anemia, idiopathic thrombocytopenia purpura, Myasthenia gravis, opsoclonus myoclonus syndrome, optic neuritis, Ord's thyroiditis, pemphigus, pernicious anaemia, polyarthritis in dogs, Reiter's syndrome, Takayasu's arteritis, warm autoimmune hemolytic anemia, fibromyalgia (FM), autoinflammatory PAPA syndrome, Familial Mediterranean Fever, polymyalgia rheumatica, polyarteritis nodosa, churg strauss syndrome; fibrosing alveolitis, hypersensitivity pneumonitis, allergic aspergillosis, cryptogenic pulmonary eosinophilia, bronchiolitis obliterans organizing pneumonia; urticaria; lupoid hepatitis; familial cold autoinflammatory syndrome, Muckle-Wells syndrome, the neonatal onset multisystem inflammatory disease, graft rejection (including allograft rejection and graft-v-host disease), otitis, chronic obstructive pulmonary disease, sinusitis, chronic prostatitis, reperfusion injury, silicosis, inflammatory myopathies, hypersensitivities and migraines. In some embodiments, an inflammatory condition is associated with an infection, e.g. viral, bacterial, fungal, parasite or prion infections. In some embodiments, an inflammatory condition is associated with an allergic response. In some embodiments, an inflammatory condition is associated with a pollutant (e.g. asbestosis, silicosis, or berylliosis).

In some embodiments, the inflammatory condition can be a local condition, e.g., a rash or allergic reaction.

In some embodiments, the inflammation is associated with a wound. In some embodiments, the technology described herein relates to methods of promoting wound healing. As used herein, “wound” refers broadly to injuries to an organ or tissue of an organism that typically involves division of tissue or rupture of a membrane (e.g., skin), due to external violence, a mechanical agency, or infectious disease. A wound can be an epithelial, endothelial, connective tissue, ocular, or any other kind of wound in which the strength and/or integrity of a tissue has been reduced, e.g. trauma has caused damage to the tissue. The term “wound” encompasses injuries including, but not limited to, lacerations, abrasions, avulsions, cuts, burns, velocity wounds (e.g., gunshot wounds), penetration wounds, puncture wounds, contusions, diabetic wounds, hematomas, tearing wounds, and/or crushing injuries. In one aspect, the term “wound” refers to an injury to the skin and subcutaneous tissue initiated in any one of a variety of ways (e.g., pressure sores from extended bed rest, wounds induced by trauma, cuts, ulcers, burns and the like) and with varying characteristics. As used herein, the term “wound healing” refers to a process by which the body of a wounded organism initiates repair of a tissue at the wound site (e.g., skin). The wounds healing process requires, in part, angiogenesis and revascularization of the wounded tissue. Wound healing can be measured by assessing such parameters as contraction, area of the wound, percent closure, percent closure rate, and/or infiltration of blood vessels as known to those of skill in the art. In some embodiments, the particles and compositions described herein can be applied topically to promote wound healing.

### Cancer

In some embodiments, the mutant semaphorin polypeptides described herein can be used to treat cancer. Immunomodulation plays an important role in the growth of cancers, and checkpoint inhibition is currently being exploited to stimulate anti-tumor immune responses. Semaphorins and mutants thereof described herein can inhibit tumor growth despite their immunosuppressive activities that inhibit, for example, transplant rejection. While not wishing to be bound by theory, rather than the indirect effect of inducing a heightened anti-tumor immune response, as shown in the Examples herein, it is likely that semaphorins and semaphorin mutants as described herein directly impact tumor cell growth, migration and function. In this regard, it is noted that semaphorin polypeptides, such as Sema3F do not necessarily kill tumor cells that express NRP-2. Rather, Sema3F, for example, collapses their f-actin cytoskeleton and inhibits their migration. Sema3F can also act upon endothelial cells, including vascular endothelial cells, inducing cytostasis, but not apoptosis; without wishing to be bound by theory, such inhibition of vascular endothelial cell growth can limit tumor growth by limiting blood supply to the tumor. This effect can permit semaphorin mutants as described herein to act upon cancers that do not express NRP-2.

In particular, the methods and compositions described herein are useful in treating cancers, including but not limited to cancers that express neuropilin 2 (NRP-2). Non-limiting examples of cancers that comprise NRP-2 expression include glioma, glioblastoma, pituitary tumors, thyroid cancer, lymphoma, lung cancer, liver cancer, carcinoid, pancreatic cancer (e.g., endocrine pancreatic tumors or pancreatic adenocarcinomas), gastric cancer, stomach cancer, colorectal cancer, acute myeloid leukemia, chronic lymphocytic leukemia B, non-small cell lung carcinoma, lung cancer, laryngeal carcinomas, laryngeal papillomas, salivary adenoid cystic carcinoma, infantile hemangiomas, bladder cancers, osteosarcomas, head & neck cancer, renal cancer, urothelial cancer, testis cancer, prostate cancer, ovarian cancer, breast cancer, cervical cancer, melanoma or metastases thereof. See e.g., Grandlement et al. “Neuropilins: A New Target for Cancer Therapy” Cancer (2011) 3:1899-1928.

In one embodiment, the subject having the tumor, cancer or malignant condition is undergoing, or has undergone, treatment with a conventional cancer therapy. In some embodiments, the cancer therapy is chemotherapy, radiation therapy, immunotherapy or a combination thereof.

In one embodiment, a semaphorin polypeptide, including a semaphorin mutant polypeptide as described herein, e.g., a Sema3F mutant polypeptide, can be used to treat cancer. While tumor inhibitory or anti-metastatic effects are likely when used alone, it is also likely that semaphorins and semaphorin mutants as described herein can enhance the efficacy of other anti-cancer agents. Exemplary anti-cancer agents that can be used in combination with a mutant semaphorin polypeptide as described herein include alkylating agents such as thiotepa and CYTOXAN™; cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues. KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas, such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN™, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK; polysaccharide complex (JHS Natural Products™, Eugene, Oreg.); razoxane; rhizoxin; sizofuran; spirogermanium; tenuazonic acid; triaziquone; 2,2′,2″-trichlorotricthylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL™ paclitaxel (Bristol-Meyers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE™ doxetaxel (Rhone-Poulenc Rorer, Antony, France); chloranbucil; GEMZAR™, gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE™, vinorelbine; novantrone; teniposide; edatrexate; daunomycin; aminopterin; xeloda; ibandronate; irinotecan (CAMPTOSAR™, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX™); lapatinib (TYKERB™): inhibitors of PKC-alpha, Raf, H-Ras, EGFR (e.g., erlotinib (TARCEVA™)) and VEGF-A that reduce cell proliferation, and pharmaceutically acceptable salts, acids or derivatives of any of the above. In addition, the methods of treatment can further include the use of radiation.

In one embodiment, the mutant semaphorin polypeptide or composition thereof can be used in combination with one or more immune checkpoint inhibitors. Exemplary immune checkpoint inhibitors include PD-L1, TIM-1, TIM-3, PD-1, CTLA4, TIGIT, LAG3, VISTA, 4-1BBL, B7-H3 and B7-DC.

### Mutant Semaphorins

Provided herein are semaphorin polypeptides with one or more mutations that permit them to be longer acting in vivo, for example, longer acting than an unmutated or wild-type semaphorin. In some embodiments, such semaphorin polypeptides comprise one or more mutations in a proproteinase cleavage motif, which prevents their cleavage by furin or other proteinases at that motif. There are two furin cleavage sites in the Sema3F proprotein molecule, the first located at amino acids 582-586 or 583-586 of human Sema3F of SEQ ID NO: 1, or at a corresponding location in Sema3F polypeptides in other species, and a second located closer to the C terminus. Other Class 3 semaphorins also include proproteinase cleavage sites in similar arrangements. At a minimum, a semaphorin mutant as described herein is mutated to inhibit cleavage at the first of such sites. Typically, such mutations are targeted such that the proproteinase cleavage motif is removed or altered such that the mutant semaphorin is resistant, at least in part, to degradation by proteinases e.g., furin. This partial or complete resistance to protease degradation at this cleavage site can increase the length of action of the mutant semaphorin polypeptides as compared to the wild-type semaphorin polypeptide from which it was derived by slowing the cleavage and degradation of the polypeptide. It is important that cleavage is not inhibited at the second, more C− terminal site, as this site can be necessary for receptor binding or activation. For Sema3F, cleavage at the first site generates a monomer protein of 65 kD. whereas cleavage at the second site generates a dimer of two 95 kD proteins (if there is no cleavage at the first). In some embodiments, part or all of the first proproteinase cleavage motif of the semaphorin (e.g., the sequence RXXR or RRSRR (SEQ ID NO: 8)) is removed or mutated. In certain embodiments, the sequence RRSRR (SEQ ID NO: 8) is inactivated by mutating the second and fourth arginines in the motif, for example, to alanine. In other embodiments, the sequence RXXR is inactivated by mutating the flanking arginines in the motif, for example, to alanine.

As one of skill in the art will appreciate, a mutation to inactivate the proproteinase cleavage motif will alter the structure, charge, or size of one or more amino acids in the motif such that the properties of the cleavage motif are disrupted and the protein is not recognized by a given proproteinase at that site. For example, mutation of one or more positively charged arginine residues to neutral alanine residues is sufficient to disrupt proproteinase recognition and cleavage at that motif. Accordingly, conservative mutations that retain a similar structure, charged, or sized amino acid residue (e.g., arginine to another positively charged amino acid residue such as lysine) are unlikely to sufficiently disrupt the proproteinase cleavage motif. Thus, mutation of one or more of the arginine residues in the (R)RXXR (e.g., RSRR (SEQ ID NO: 14), or RRSRR (SEQ ID NO: 8)) to a negatively charged residue (e.g., aspartic acid or glutamic acid) or a neutral amino acid residue (e.g., alanine, cysteine, glycine, isoleucine, leucine, methionine, phenylalanine, proline, valine) is specifically contemplated herein. In certain embodiments, at least two of the arginine residues in the (R)RXXR motif are mutated to a negatively charged or neutral amino acid residue.

In one embodiment, the mutant semaphorin polypeptide comprises an in vivo half-life that is at least 6 h longer than the in vivo half-life of the wild-type semaphorin polypeptide from which it is derived. In other embodiments, the mutant semaphorin polypeptide comprises an in vivo half-life that is at least 7 h, at least 8 h, at least 9 h, at least 10 h, at least 11 h, at least 12 h, at least 14 h, at least 16 h, at least 18 h, at least 20 h, at least 22 h, at least 24 h, at least 30 h, at least 36 h, at least 48 h, at least 72 h, or longer than the in vivo half-life of the wild-type semaphorin polypeptide from which it is derived. As will be appreciated by one of skill in the art, the in vivo half-life can be determined by measuring the concentration of e.g., the mutant semaphorin polypeptide in a given biological sample (e.g., blood), for example, every hour, until the concentration of the mutant semaphorin polypeptide is approximately half of the peak concentration of the polypeptide following administration. The half-life can be assessed after a single dose or alternatively, can be measured once the measured concentration of the multiply delivered polypeptide has reached steady-state kinetics.

Mutant semaphorin polypeptides can be designed from any desired semaphorin polypeptide including, but not limited to, SEMA 3A polypeptide, SEMA 3C polypeptide, SEMA 3F polypeptide or SEMA 3G polypeptide (see Table 1 for exemplary human sequences). In some embodiments, the semaphorin polypeptide to be mutated is a human semaphorin polypeptide. For example, a “Sema3F polypeptide” can include the human polypeptide (SEQ ID NO: 1, NCBI Ref Seq: NP_004177) as well as homologs from other species, including but not limited to bovine, dog, cat chicken, murine, rat, porcine, ovine, turkey, horse, fish, baboon and other primates.

TABLE 1
Exemplary Human Semaphorin 3 polypeptide Sequences

| | | UniProt/Swiss | SEQ ID |
|---|---|---|---|
| Semaphorin | Sequence | Prot ID No. | NO. |
| SEMA 3A | mgwltrivcl fwgvlltara nyqngknnvp | Q14563.1 | 5 |
| | rlklsykeml esnnvitfng lansssyhtf | | |
| | lldeersrly vgakdhifsf dlvnikdfqk | | |
| | ivwpvsytrr deckwagkdi lkecanfikv | | |
| | lkayngthly acgtgafhpi ctyieighhp | | |
| | ednifklens hfengrgksp ydpklltasl | | |
| | lidgelysgt aadfmgrdfa ifrtlghhhp | | |
| | irteqhdsrw lndpkfisah lisesdnped | | |
| | dkvyfffren aidgehsgka tharigqick | | |
| | ndfgghrslv nkwttflkar licsvpgpng | | |
| | idthfdelqd vflmnfkdpk npvvygvftt | | |
| | ssnifkgsav cmysmsdvrr vflgpyahrd | | |
| | gpnyqwvpyq grvpyprpgt cpsktfggfd | | |
| | stkdlpddvi tfarshpamy npvfpmnnrp | | |
| | iviktdvnyq ftqivvdrvd aedgqydvmf | | |
| | igtdvgtvlk vvsipketwy dleevlleem | | |
| | tvfreptais amelstkqqq lyigstagva | | |
| | qlplhrcdiy gkacaeccla rdpycawdgs | | |
| | acsryfptak rrtrrqdirn gdplthcsdl | | |
| | hhdnhhghsp eeriiygven sstflecspk | | |
| | sqralvywqf qrrneerkee irvddhiirt | | |
| | dqglllrslq qkdsgnylch avehgfigtl | | |
| | lkvtlevidt ehleellhkd ddgdgsktke | | |
| | msnsmtpsqk vwyrdfmqli nhpnlntmde | | |
| | fceqvwkrdr kqrrqrpght pgnsnkwkhl | | |
| | qenkkgrnrr theferaprs v | | |
| SEMA 3C | mafrticvlv gvficsicvk gssqpqarvy | Q99985.2 | 6 |
| | ltfdelretk tseyfslshh pldyrillmd | | |
| | edqdriyvgs kdhilslnin nisqealsvf | | |
| | wpastikvee ckmagkdpth gcgnfvrviq | | |
| | tfnrthlyvc gsgafspvct ylnrgrrsed | | |
| | qvfmidskce sgkgrcsfnp nvntvsvmin | | |
| | eelfsgmyid fmgtdaaifr sltkrnavrt | | |
| | dqhnskwlse pmfvdahvip dgtdpndakv | | |
| | yfffkekltd nnrstkqihs miaricpndt | | |
| | gglrslvnkw ttflkarlvc svtdedgpet | | |
| | hfdeledvfl letdnprttl vygifttsss | | |
| | vfkgsavcvy hlsdiqtvfn gpfahkegpn | | |
| | hglisyggri pyprpgtcpg gaftpnmrtt | | |
| | kefpddvvtf irnhplmyns iypihkrpli | | |
| | vrigtdykyt kiavdrvnaa dgryhvlflg | | |
| | tdrgtvqkvv vlptnnsysg elileelevf | | |
| | knhapittmk isskkqqlyv ssnegvsqvs | | |
| | lhrchiygta cadcclardp ycawdghscs | | |
| | rfyptgkrrs rrqdvrhgnp ltqcrgfnlk | | |
| | ayrnaaeivq ygvknnttfl ecapkspqas | | |
| | ikwllqkdkd rrkevklner iiatsqglli | | |
| | rsvggsdqgl yhciatensf kgtiakinfk | | |
| | vldsemvavv tdkwspwtwa ssvralpfhp | | |
| | kdimgafshs emqminqyck dtrqqhqqgd | | |
| | esqkmrgdyg klkalinsrk srnrrnqlpe s | | |
| SEMA 3F | mlvaglllwa slltgawpsf ptqdhlpatp | Q13275.2 | 1 |
| | rvrlsfkelk atgtahffnf llnttdyril | | |
| | lkdedhdrmy vgskdyvlsl dlhdinrepl | | |
| | iihwaaspqr ieecvlsgkd vngecgnfvr | | |
| | liqpwnrthl yvcgtgaynp mctyvnrgrr | | |
| | aqatpwtqtq avrgrgsrat dgalrpmpta | | |
| | prqdyifyle perlesgkgk cpydpkldta | | |
| | salineelya gvyidfmgtd aaifrtlgkq | | |
| | tamrtdqyns rwlndpsfih aelipdsaer | | |
| | nddklyfffr ersaeapqsp avyarigric | | |
| | lnddgghccl vnkwstflka rlvcsvpged | | |
| | giethfdelq dvfvqqtqdv rnpviyavft | | |
| | ssgsvfrgsa vcvysmadir mvfngpfahk | | |
| | egpnyqwmpf sgkmpyprpg tcpggtftps | | |
| | mkstkdypde vinfmrshpl myqavyplqr | | |
| | rplvvrtgap yrlttiavdq vdaadgryev | | |
| | lflgtdrgtv qkvivlpkdd qeleelmlee | | |
| | vevfkdpapv ktmtisskrq qlyvasavgv | | |
| | thls1hrcqa ygaacadccl ardpycawdg | | |
| | qacsrytass krrsrrqdvr hgnpirgcrg | | |
| | fnsnanknav esvqygvags aaflecqprs | | |
| | pqatvkwlfq rdpgdrrrei raedrflrte | | |
| | qglllralql sdrglyscta tennfkhvvt | | |
| | rvqlhvlgrd avhaalfppl smsappppga | | |
| | gpptppygel aqllaqpevg lihqycqgyw | | |
| | rhvppsprea pgaprspepq dqkkprnrrh hppdt | | |
| SEMA 3G | mapsawaicw llgglllhgg ssgpspgpsv | Q9NS98.1 | 7 |
| | prlrlsyrdl lsanrsaifl gpqgslnlqa | | |
| | myldeyrdrl flggldalys lrldqawpdp | | |
| | revlwppqpg qreecvrkgr dpltecanfv | | |
| | rvlqphnrth llacgtgafq ptcalitvgh | | |
| | rgehvlhlep gsvesgrgrc phepsrpfas | | |
| | tfidgelytg ltadflgrea mifrsggprp | | |
| | alrsdsdqsl lhdprfvmaa ripensdqdn | | |
| | dkvyfffset vpspdggsnh vtvsrvgrvc | | |
| | vndaggqrvl vnkwstflka rlvcsvpgpg | | |
| | gaethfdqle dvfllwpkag kslevyalfs | | |
| | tvsavfqgfa vcvyhmadiw evfngpfahr | | |
| | dgpqhqwgpy ggkvpfprpg vcpskmtaqp | | |
| | grpfgstkdy pdevlqfara hplmfwpvrp | | |
| | rhgrpvlvkt hlaqqlhqiv vdrveaedgt | | |
| | ydviflgtds gsvlkvialq aggsaepeev | | |
| | vleelqvfkv ptpitemeis vkrqmlyvgs | | |
| | rlgvaqlrlh qcetygtaca ecclardpyc | | |
| | awdgascthy rpslgkrrfr rqdirhgnpa | | |
| | lgclgqsqee eavglvaatm vygtehnstf | | |
| | leclpkspqa avrwllqrpg degpdqvktd | | |
| | ervlhtergl lfrrlsrfda gtytcttleh | | |
| | gfsqtvvrla lvvivasqld nlfppepkpe | | |
| | eppargglas tppkawykdi lqligfanlp | | |
| | rvdeycervw crgttecsgc frsrsrgkqa | | |
| | rgkswaglel gkkmksrvha ehnrtpreve at | | |

*bolded text indicates a minimal proproteinase consensus sequence while underlined text indicates a larger proproteinase consensus sequence to be inactivated by mutation.

In order to retain the therapeutic benefit of a semaphorin polypeptide, it is specifically contemplated herein that the mutant semaphorin polypeptide retains the ability to bind to neuropilin 1 or 2 (NRP-1 or NRP-2). In other embodiments, the mutant semaphorin polypeptide retains at least 50% of the activity of the wild-type semaphorin polypeptide as assessed by measuring (i) binding to NRP-1 or NRP-2, and/or (ii) activation of effectors downstream of NRP-1 or NRP-2, and/or (iii) immunomodulation. In other embodiments, the mutant semaphorin polypeptide retains at least 50% of the activity of the wild-type semaphorin polypeptide as assessed by measuring suppression of allograft rejection, e.g., as measured in an appropriate animal model In other embodiments, the mutant semaphorin polypeptide retains at least 60%, at least 70%, at least 75%, at least 80%, at least 90%, at least 95%, at least 99%, activity or retains activity that is at least substantially similar to the wild-type semaphorin polypeptide from which it is derived. It is further contemplated herein that the mutant semaphorin can comprise increased activity over the wild-type semaphorin, for example, at least 2-fold, at least 5-fold, at least 10-fold, at least 50-fold, at least 100-fold, at least 1000-fold or more activity compared to the wild-type semaphorin polypeptide.

In one embodiment, a mutant semaphorin polypeptide as described herein binds in the same binding site of the NRP-2 receptor as a wild-type semaphorin polypeptide. The binding site of wild-type semaphorin polypeptides and domain interactions with the NRP-2 receptor is described in e.g., Appleton et al. The EMBO J (2007) 26:49012-4912. Amino acid insertions and deletions are specifically contemplated herein in a mutant semaphorin polypeptide provided that such insertions or deletions do not impair binding of the mutant semaphorin polypeptide to the NRP-2 receptor. In certain embodiments, an insertion comprises at least one additional residue but does not exceed 20 additional residues, for example, 1-18 residues, 1-16 residues, 1-15 residues, 1-14 residues, 1-12 residues, 1-10 residues, 1-9 residues, 1-8 residues, 1-7 residues, 1-6 residues, 1-5 residues, 1-4 residues, 1-3 residues or 1-2 residues are inserted. In other embodiments, a deletion comprises removal of at least one residue but does not exceed 10 residues, for example, less than 9, less than 8, less than 7, less than 6, less than 5, less than 4, less than 3, or 2 residues are deleted.

In one embodiment, a mutant semaphorin polypeptide as described herein, at a minimum, binds to the “a” domain (e.g., a1/a2 domain(s)) of the NRP-2 receptor.

In one embodiment, a mutant semaphorin polypeptide described herein comprises 10 or fewer, 9 or fewer, 8 or fewer, 7 or fewer, 6 or fewer, 5 or fewer, 4 or fewer, 3 or fewer, 2 or fewer, 1 or fewer, i.e., does not comprise any mutations, including substitutions, deletions or insertions, outside of the proproteinase cleavage site.

It is also contemplated herein that additional mutations can be introduced (e.g., outside of the proproteinase cleavage site motif), for example, to further improve in vivo half-life, enhance activity, or reduce side effects in a subject. Conservative substitution variants that maintain the desired activity of a mutant semaphorin polypeptide can include a conservative substitution as defined herein. The identification of amino acids most likely to be tolerant of conservative substitution while maintaining at least 50% of the activity of the wild-type is guided by, for example, sequence alignment with semaphorin (e.g., SEMA 3A, SEMA 3C, SEMA 3F, SEMA 3G, etc.) homologs or paralogs from other species. Amino acids that are identical between such homologs are less likely to tolerate change, while those showing conservative differences are obviously much more likely to tolerate conservative change in the context of an artificial variant. Similarly, positions with non-conservative differences are less likely to be critical to function and more likely to tolerate conservative substitution in an artificial variant. Variants, fragments, and/or fusion proteins can be tested for activity, for example, by administering the variant to an appropriate animal model of allograft rejection as described herein. Further discussion of the structure of Sema3F and NRP-2 can be found, e.g. in Klagsbrun M, Eichmann A, Cytokine Growth Factor Rev, 2005; which is incorporated by reference herein in its entirety.

In one embodiment, an additional mutation outside of the proproteinase cleavage motif in a given mutant semaphorin polypeptide is designed such that the resulting mutant semaphorin retains the ability to bind and activate NRP-2. For example, additional mutations within the N-terminal sema domain, which is important for binding NRP-2, should comprise conservative amino acid substitutions only and preferably not affect the binding and activation of NRP-2. Similarly, if non-conservative mutations are desired, such mutations can be made outside of the sema domain necessary for NRP-2 binding.

In some embodiments, a polypeptide, e.g., a mutant semaphorin polypeptide as described herein, can be a variant of a sequence described herein, e.g. a variant of a mutant semaphorin polypeptide lacking the proproteinase cleavage site sequence RRSRR (SEQ ID NO: 8). In some embodiments, the variant is a conservative substitution variant. Variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Polypeptide-encoding DNA sequences encompass sequences that comprise one or more additions, deletions, or substitutions of nucleotides when compared to a native or reference DNA sequence, but that encode a variant protein or fragment thereof that retains the relevant biological activity relative to the reference protein, e.g., can suppress allograft rejection at least 50% as well as its corresponding mutant semaphorin polypeptide or the wild-type semaphorin from which it is derived. As to amino acid sequences, one of skill will recognize that individual substitutions, deletions or additions to a nucleic acid, peptide, polypeptide, or protein sequence which alters a single amino acid or a small percentage, (i.e. 5% or fewer, e.g. 4% or fewer, or 3% or fewer, or 1% or fewer) of amino acids in the encoded sequence is a “conservatively modified variant” where the alteration results in the substitution of an amino acid with a chemically similar amino acid. It is contemplated that some changes can potentially improve the relevant activity, such that a variant, whether conservative or not, has more than 100% of the activity of e.g., the mutant semaphorin polypeptide or the wild-type semaphorin polypeptide from which it is derived, e.g. 110%, 125%, 150%, 175%, 200%, 500%, 1000% or more.

One method of identifying amino acid residues which can be substituted is to align, for example, human Sema3F to a Sema3F homolog from one or more non-human species. Alignment can provide guidance regarding not only residues likely to be necessary for function but also, conversely, those residues likely to tolerate change. Where, for example, an alignment shows two identical or similar amino acids at corresponding positions, it is more likely that that site is important functionally. Where, conversely, alignment shows residues in corresponding positions to differ significantly in size, charge, hydrophobicity, etc., it is more likely that that site can tolerate variation in a functional polypeptide. The variant amino acid or DNA sequence can be at least 90%, at least 95%, at least 96%, at least 97%, at least 98%, at least 99%, or more, identical to a native or reference sequence, e.g., SEQ ID NO: 1 or a nucleic acid encoding one of those amino acid sequences. The degree of homology (percent identity) between a native and a mutant sequence can be determined, for example, by comparing the two sequences using freely available computer programs commonly employed for this purpose on the world wide web. The variant amino acid or DNA sequence can be at least 90%, at least 91%, at least 92%, at least 93%, at least 94%, at least 95%, at least 96%, at least 97%, at least 98%, at least 99%, or more, similar to the sequence from which it is derived (referred to herein as an “original” sequence). The degree of similarity (percent similarity) between an original and a mutant sequence can be determined, for example, by using a similarity matrix. Similarity matrices are well known in the art and a number of tools for comparing two sequences using similarity matrices are freely available online, e.g. BLASTp (available on the world wide web at http://blast.ncbi.nlm.nih.gov), with default parameters set.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity of a native or reference polypeptide is retained. Conservative substitution tables providing functionally similar amino acids are well known in the art. Such conservatively modified variants are in addition to and do not exclude polymorphic variants, interspecies homologs, and alleles consistent with the disclosure. Typically conservative substitutions for one another include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)).

Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

In some embodiments, a polypeptide, e.g., a mutant semaphorin polypeptide, administered to a subject can comprise one or more amino acid substitutions or modifications beyond the mutations to the proproteinase cleavage motif that increases the half-life of the mutant semaphorin. In some embodiments, the substitutions and/or modifications can prevent or reduce proteolytic degradation and/or prolong half-life of the polypeptide in the subject. In some embodiments, a polypeptide can be modified by conjugating or fusing it to other polypeptide or polypeptide domains such as, by way of non-limiting example, transferrin (WO06096515A2), albumin (Yeh et al., 1992), growth hormone (US2003104578AA); cellulose (Levy and Shoseyov, 2002); and/or Fc fragments (Ashkenazi and Chamow, 1997). The references in the foregoing paragraph are incorporated by reference herein in their entireties.

In some embodiments, a polypeptide, e.g., a mutant semaphorin, as described herein can comprise at least one peptide bond replacement. A mutant SEMA polypeptide as described herein can comprise one type of peptide bond replacement or multiple types of peptide bond replacements, e.g. 2 types, 3 types, 4 types, 5 types, or more types of peptide bond replacements. Non-limiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, olefinic group, and derivatives thereof.

In some embodiments, a polypeptide, e.g., a mutant semaphorin polypeptide, as described herein can comprise naturally occurring amino acids commonly found in polypeptides and/or proteins produced by living organisms, e.g. Ala (A), Val (V), Leu (L), Ile (I), Pro (P), Phe (F), Trp (W), Met (M), Gly (G), Ser(S), Thr (T), Cys (C), Tyr (Y), Asn (N), Gln (Q), Asp (D), Glu (E), Lys (K), Arg (R), and His (H). In some embodiments, a Sema3F polypeptide as described herein can comprise alternative amino acids. Non-limiting examples of alternative amino acids include, D-amino acids; beta-amino acids; homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citrulinc, alpha-methyl-alanine, para-benzoylphenylalanine, para-amino phenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-l-cyclopentanecarboxylic acid, 1-amino-l-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof.

In some embodiments, a polypeptide, e.g. a mutant semaphorin polypeptide, can be modified, e.g. by addition of a moiety to one or more of the amino acids that together comprise the peptide. In some embodiments, a polypeptide as described herein can comprise one or more moiety molecules, e.g., 1 or more moiety molecules per polypeptide, 2 or more moiety molecules per polypeptide, 5 or more moiety molecules per polypeptide, 10 or more moiety molecules per polypeptide or more. In some embodiments, a polypeptide as described herein can comprise one or more types of modifications and/or moieties, e.g. 1 type of modification, 2 types of modifications, 3 types of modifications or more types of modifications. Non-limiting examples of modifications and/or moieties include PEGylation; glycosylation; HESylation; ELPylation; lipidation; acetylation; amidation; end-capping modifications; cyano groups; phosphorylation; albumin, and cyclization. In some embodiments, an end-capping modification can comprise acetylation at the N-terminus, N-terminal acylation, and N-terminal formylation. In some embodiments, an end-capping modification can comprise amidation at the C-terminus, introduction of C-terminal alcohol, aldehyde, ester, and thioester moieties. The half-life of a mutant polypeptide can be further increased by the addition of moieties, e.g. PEG, albumin, or other fusion partners (e.g. Fc fragment of an immunoglobin).

Alterations of the original amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations include those disclosed by Khudyakov et al. “Artificial DNA: Methods and Applications” CRC Press, 2002; Braman “In Vitro Mutagenesis Protocols” Springer, 2004; and Rapley “The Nucleic Acid Protocols Handbook” Springer 2000; which are herein incorporated by reference in their entireties. In some embodiments, a polypeptide as described herein can be chemically synthesized and mutations can be incorporated as part of the chemical synthesis process.

In some embodiments, a mutant SEMA polypeptide can be a polypeptide that can bind a Sema3F receptor, e.g. NRP-2. In some embodiments, a mutant semaphorin polypeptide can be a Sema3F polypeptide that can bind a domain of NRP-2 selected from the group consisting of the A1; the A2; the B1; and the B2 domain. In other embodiments, a mutant semaphorin polypeptide is a polypeptide that binds NRP-1.

The polypeptides of the present invention can be synthesized using well known methods including recombinant methods and chemical synthesis. Recombinant methods of producing a polypeptide through the introduction of a vector including nucleic acid encoding the polypeptide into a suitable host cell are well known in the art, e.g., as described in Sambrook et al., Molecular Cloning: A Laboratory Manual, 2d Ed, Vols 1 to 8, Cold Spring Harbor, NY (1989); M.W. Pennington and B.M. Dunn, Methods in Molecular Biology: Peptide Synthesis Protocols, Vol 35, Humana Press, Totawa, NJ (1994), contents of both of which are herein incorporated by reference. Peptides can also be chemically synthesized using methods well known in the art. See for example, Merrifield et al., J. Am. Chem. Soc. 85:2149 (1964); Bodanszky, M., Principles of Peptide Synthesis, Springer-Verlag, New York, NY (1984); Kimmerlin, T. and Seebach, D. J. Pept. Res. 65:229-260 (2005); Nilsson et al., Annu. Rev. Biophys. Biomol. Struct. (2005) 34:91-118; W.C. Chan and P.D. White (Eds.) Fmoc Solid Phase Peptide Synthesis: A Practical Approach, Oxford University Press, Cary, NC (2000); N.L. Benoiton, Chemistry of Peptide Synthesis, CRC Press, Boca Raton, FL (2005); J. Jones, Amino Acid and Peptide Synthesis, 2^{nd} Ed, Oxford University Press, Cary, NC (2002); and P. Lloyd-Williams, F. Albericio, and E. Giralt, Chemical Approaches to the synthesis of peptides and proteins, CRC Press, Boca Raton, FL (1997), contents of all of which are herein incorporated by reference. Peptide derivatives can also be prepared as described in U.S. Pat. Nos. 4,612,302; 4,853,371; and 4,684,620, and U.S. Pat. App. Pub. No. 2009/0263843, contents of all which are herein incorporated by reference.

### Nucleic Acids and Vectors

In some embodiments, the technology described herein relates to a nucleic acid encoding a mutant semaphorin polypeptide as described herein. As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. Alternatively, it can be a single-stranded nucleic acid not derived from any double-stranded DNA. In one aspect, the template nucleic acid is DNA. In another aspect, the template is RNA. Suitable nucleic acid molecules include DNA, including genomic DNA or cDNA. Other suitable nucleic acid molecules include RNA, including mRNA. The nucleic acid molecule can be naturally occurring, as in genomic DNA, or it can be synthetic, i.e., prepared based upon human action, or can be a combination of the two. The nucleic acid molecule can also have certain modification(s) such as 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA), cholesterol addition, and phosphorothioate backbone as described in U.S. Patent Application 20070213292; and certain ribonucleoside that are linked between the 2′-oxygen and the 4′-carbon atoms with a methylene unit as described in U.S. Pat. No. 6,268,490, wherein both patent and patent application are incorporated herein by reference in their entirety.

In some embodiments, a nucleic acid encoding a mutant semaphorin polypeptide as described herein is comprised by a vector. In some of the aspects described herein, a nucleic acid sequence encoding a mutant semaphorin polypeptide as described herein is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc. Expression vectors useful for the preparation of mutant Sema3F polypeptides as described herein include, for example, the pSecTag vectors (e.g., pSecTagA, pSecTagB, pSecTagC, pSecTag2A, pSecTag 2B, pSecTag2C) and the pcDNA3.1+ vectors (e.g., pcDNA3.1+/C-His, pcDNA3.1+/N-His and pcDNA3.1/HisA), available from Thermo Fisher, among others.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector can comprise additional elements, for example, the expression vector can have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain a nucleic acid encoding a mutant semaphorin polypeptide as described herein in place of non-essential viral genes. The vector and/or particle can be utilized for the purpose of transferring nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

### Pharmaceutically Acceptable Carriers

In some embodiments, the technology described herein relates to a pharmaceutical composition as described herein, and optionally a pharmaceutically acceptable carrier. Pharmaceutically acceptable carriers and diluents include saline, aqueous buffer solutions, solvents and/or dispersion media. Polypeptides, such as a mutant semaphorin polypeptide as described herein, will generally be formulated for parenteral administration and can be combined with any carrier suited for parenteral routes of administration. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) scrum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent as described herein.

In some embodiments, the pharmaceutical composition as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration to a patient, including, but not limited to, DUROS®-type dosage forms and dose-dumping.

Suitable vehicles that can be used to provide parenteral dosage forms are well known to those skilled in the art. Examples include, without limitation: sterile water; water for injection USP; saline solution; glucose solution; aqueous vehicles such as but not limited to, sodium chloride injection, Ringer's injection, dextrose injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of a composition as disclosed herein can also be incorporated into the parenteral dosage forms of the disclosure, including conventional and controlled-release parenteral dosage forms.

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled-or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos.: 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

### Dosage, Administration and Efficacy

In some embodiments, the methods described herein relate to treating a subject having or diagnosed as having, e.g. an inflammatory condition with a mutant semaphorin polypeptide or compositions thereof, as described herein. Subjects having, e.g. an inflammatory condition can be identified by a physician using current methods of diagnosis. Symptoms and/or complications of, e.g. inflammatory conditions which characterize these conditions and aid in diagnosis are well known in the art and include but are not limited to, elevated levels of immune response markers, swelling, and/or heat. A family history of an inflammatory condition or exposure to risk factors for an inflammatory condition can also aid in determining if a subject is likely to have the inflammatory condition or in making a diagnosis of a particular inflammatory condition.

The compositions and methods described herein can be administered to a subject having or diagnosed as having, e.g. an inflammatory condition or being in need of immunosuppression (e.g. having received an allograft or transplant). In some embodiments, the methods described herein comprise administering an effective amount a composition described herein, to a subject in order to alleviate a symptom of, e.g. an inflammatory condition. As used herein, “alleviating a symptom” is ameliorating a condition or symptom associated with the condition. As compared with an equivalent untreated control, such reduction is by at least 10% as measured by any standard technique. A variety of means for administering the compositions described herein to subjects are known to those of skill in the art. Such methods can include, but are not limited to parenteral, intravenous, intramuscular, subcutaneous, transdermal, airway (aerosol), pulmonary, cutaneous, topical, or injection administration. Administration can be local or systemic.

The term “effective amount” as used herein refers to the amount of a composition needed to alleviate at least one or more symptom of the disease or disorder, and relates to a sufficient amount of pharmacological composition to provide the desired effect. The term “therapeutically effective amount” therefore refers to an amount that is sufficient to provide a particular effect when administered to a typical subject. An effective amount as used herein, in various contexts, would also include an amount sufficient to delay the development of a symptom of the disease, alter the course of a disease symptom (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and/or the ED50) (the dose therapeutically effective in 50% of the population) as appropriate. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of a composition, which achieves a half-maximal inhibition of symptoms) as determined in cell culture, or in an appropriate animal model. Levels in plasma can be measured, for example, by immunoassay or chromatography. The effects of any particular dosage can be monitored by a suitable bioassay, e.g., assay for immune responsiveness, among others. The dosage can be determined by a physician and adjusted, as necessary, to suit observed effects of the treatment.

In one embodiment, the therapeutically effective amount is an amount of a pharmaceutical composition, as described herein, to reduce the level of a biomarker that predicts early stage organ rejection by at least 20% (e.g., at least 30%, at least 40%, at least 50%, at least 60%, at least 70%, at least 80%, at least 90%, at least 95%, at least 99%, or the absence of the biomarker using standard means of detection). Such biomarkers can be specific to the organ that has been transplanted (e.g., creatinine to monitor kidney function) or can be non-specific markers of chronic inflammation, such as pro-inflammatory cytokines or c-reactive protein. In some embodiments, the biomarker is present in an exosome sample from the subject. In one embodiment, the therapeutically effective amount of a pharmaceutical composition as described herein is an amount sufficient to reduce the expression of one or more inflammatory markers by at least 20% (e.g., at least 30%, at least 40%, at least 50%, at least 60%, at least 70%, at least 80%, at least 90%, at least 95%, at least 99%, or the absence of the biomarker using standard means of detection). Non-limiting examples of inflammatory markers include cytokines/chemokines (e.g., IL-1α, IL-1β, IL-2, IL-6, Il-8, IL-12, TNF-α, IFN-γ), immune related effectors (e.g., white blood cell count, neutrophil:lymphocyte ration, platelet:lymphocyte ratio, Glasgow prognostic score etc.), acute phase proteins (e.g., C-reactive protein, serum amyloid A etc.), reactive oxygen species, reactive nitrogen species, 3-nitrotyrosine, oxidatively/nitrosatively modified DNA or proteins, prostaglandins, cyclooxygenase-related factors (e.g., COX-2 expression), and transcription factors or growth factors associated with inflammatory effects (e.g., NF-κB activation, STAT3 activation etc.) See, for example, Brenner et al. Cancer Epidemiol Biomarkers Prev (2014) 23(9):1729-1751.

In some embodiments, a mutant semaphorin polypeptide or composition thereof as described herein can be administered intravenously. In some embodiments, such mutant semaphorin polypeptides can be administered intramuscularly, subcutaneously, or intradermally. In some embodiments, a mutant semaphorin polypeptide can be administered locally to a site of inflammation.

The methods described herein can further comprise administering a second agent and/or treatment to the subject, e.g. as part of a combinatorial therapy. By way of non-limiting example, if a subject is to be treated for inflammation according to the methods described herein, the subject can also be administered a second agent and/or treatment known to be beneficial for subjects suffering from pain or inflammation. Examples of such agents and/or treatments include, but are not limited to, non-steroidal anti-inflammatory drugs (NSAIDs—such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; or opiates (e.g. endorphins, enkephalins, and dynorphin), steroids, analgesics, barbiturates, oxycodone, morphine, lidocaine, and the like. In some embodiments, the additional anti-inflammatory agent can be a steroid (e.g., a corticosteroid or glucocorticoid); a calcineurin inhibitor (e.g. cyclosporine, tacrolimus, pimecrolimus, or FK506); an mTOR inhibitor (e.g., everolimus, temsirolimus, rapamycin, deforolimus, TOP216, OSI-027, TAFA93, nab-rapamycin, tacrolimus, biolimus, CI-779, ABT-578, AP-23675, BEZ-235, QLT-0447, ABI-009, BC-210, salirasib, AP-23841, AP-23573, KU-0059475, 32-deoxorapamycin, 16-pent-2-ynyloxy-32-deoxorapamycin, 16-pent-2-ynyloxy-32 (S or R)-dihydro-rapamycin, 16-pent-2-ynyloxy-32 (S or R)-dihydro-40-O-(2-hydroxyethyl)-rapamycin, 40-O-(2-hydroxyethyl)-rapamycin, 32-deoxorapamycin; 16-pent-2-ynyloxy-32(S)-dihydrorapamycin; socalledrapalogs; AP23464; PI-103, PP242, PP30, Torin1; and derivatives or pharmaceutically acceptable salts thereof as well as and compounds described in, e.g. U.S. Patent Publications 2011/0178070; 2011/0021515; 2007/0112005; 2011/0054013; International Patent Publications WO98/02441; WO01/14387; WO99/15530; WO07/135411; WO03/64383; WO96/41807; WO95/16691; WO94/09010; European Patent No. EP1880723; and U.S. Pat. Nos. 8,163,775; 6,329,386; 6,200,985; 6,117,863; 6,015,815; 6,015,809; 6,004,973; 5,985,890; 5,955,457; 5,922,730; 5,912,253; 5,780,462; 5,665,772; 5,637,590; 5,567,709; 5,563,145; 5,559,122; 5,559,120; 5,559,119; 5,559,112; 5,550,133; 5,541,192; 5,541,191; 5,532,355; 5,530,121; 5,530,007; 5,525,610; 5,521,194; 5,519,031; 5,516,780; 5,508,399; 5,508,290; 5,508,286; 5,508,285; 5,504,291; 5,504,204; 5,491,231; 5,489,680; 5,489,595; 5,488,054; 5,486,524; 5,486,523; 5,486,522; 5,484,791; 5,484,790; 5,480,989; 5,480,988; 5,463,048; 5,446,048; 5,434,260; 5,411,967; 5,391,730; 5,389,639; 5,385,910; 5,385,909; 5,385,908; 5,378,836; 5,378,696; 5,373,014; 5,362,718; 5,358,944; 5,346,893; 5,344,833; 5,302,584; 5,262,424; 5,262,423; 5,260,300; 5,260,299; 5,233,036; 5,221,740; 5,221,670; 5,202,332; 5,194,447; 5,177,203; 5,169,851; 5,164,399; 5,162,333; 5,151,413; 5,138,051; 5,130,307; 5,120,842; 5,120,727; 5,120,726; 5,120,725; 5,118,678; 5,118,677; 5,100,883; 5,023,264; 5,023,263; and 5,023,262; which are incorporated by reference herein in their entireties.); rapamycin (sirolimus) or an analogue thereof (e.g. everolimus, temsirolimus, ridaforolimus, deforolimus); or an anti-proliferative agent (e.g. mycophenoloate moefitil, azathioprine). In some embodiments, the mTOR inhibitor can be rapamycin or an analogue thereof, e.g. everolimus, temsirolimus, ridaforolimus, or deforolimus. Anti-proliferative agents can include, by way of non-limiting example, alkylating agents (e.g. cyclophosphamide, platinum compounds, and nitrosoureas), antimetabolites (e.g. methotrexate, azathioprine, mercaptopurine, fluorouracil, etc), and cytotoxic antibiotics (e.g., dactinomycin, anthracyclines, mitomycin C, bleomycin, and mithramycin).

In certain embodiments, an effective dose of a composition as described herein can be administered to a patient once. In certain embodiments, an effective dose of a composition can be administered to a patient repeatedly (e.g., at least twice). For systemic administration, subjects can be administered a therapeutic amount of a composition, such as, e.g. 1 μg/k, 10 μg/kg, 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. a marker of an immune response by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active ingredient. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition can be administered over a period of time, such as over a 5 minute, 10 minute, 15 minute, 20 minute, or 25 minute period.

The dosage ranges for the administration of a composition, according to the methods described herein depend upon, for example, the form of the active ingredient, its potency, and the extent to which symptoms, markers, or indicators of a condition described herein are desired to be reduced, for example the percentage reduction desired for an immune response or the extent to which, for example, an immune response is desired to be induced. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a composition in, e.g. the treatment of a condition described herein, or to induce a response as described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. graft rejection. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing or slowing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of allograft rejection in mice. When using an experimental animal model, efficacy of treatment is evidenced when a statistically significant change in a marker is observed, e.g. the level and/or proliferation of activated T or B cells.

In vitro and animal model assays are provided herein which allow the assessment of a given dose of a composition described herein, e.g. a mutant semaphorin polypeptide or composition thereof. By way of non-limiting example, the effects and dose response of a composition can be assessed by treating CD4+ T cells with mitogen (anti-CD3) in the presence and absence of the composition and measuring proliferation and/or the production of cytokines including, but not limited to, IL-2, IL-4 IFN-gamma, IL-17, IL-10, IL-15 and others, where Neuropilin-2 activity is indicated by a lower level of proliferation and/or decreased production of select and/or programs of cytokines.

The efficacy of a given dosage combination can also be assessed in an animal model, e.g. a mouse model of allograft rejection, colitis, or skin inflammation/delayed type hypersensitivity (DTH). For example, C57BL/6 mice can be the recipients of a cardiac or skin allograft from BALB/c mice. Rejection and/or survival can be monitored, e.g. over at least 1-3 weeks. In DTH, skin swelling can be monitored over 1-7 days. It is contemplated herein that treatment of allograft recipients with one or more mutant semaphorin polypeptides described herein inhibits allograft rejection. It is further contemplated herein that treatment with a mutant semaphorin polypeptide or composition thereof reduces both the inflammatory response, and DTH responses.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

As used herein, “suppression of the immune system” refers to decreasing or inhibiting the immune function of an animal, as measured by any parameter of the various immune functions of the immune system. Non-limiting examples of parameters of immune function can include the magnitude of the antibody response, the response of a B cell, the response of a T cell, the proliferation of T cells, the production of immunomodulatory cytokines, and/or the response to an antigen (e.g. to allogenic or xenogenic cells). Conversely, “stimulation of the immune system” refers to an increase or activation of the immune function of an animal, as measured by any parameter of the various immune functions of the immune system.

As used herein, “graft rejection” or “transplant rejection” refers to any immunologically mediated hyperacute, acute, or chronic injury to a tissue or organ derived from a source other than the host. The term thus encompasses both cellular and antibody-mediated rejection, as well as rejection of both allografts and xenografts.

In some embodiments, suppressing the immune system can comprise suppressing graft vs, host disease. “Graft-versus-host disease” (GVHD) is a reaction of donated tissue against a patient's own tissue. GVHD is seen most often with bone marrow transplant, but can occur with the transplant of other tissues or cells. GVHD is seen most often in cases where the tissue donor is unrelated to the patient or when the donor is related to the patient but not a perfect match. There are two forms of GVHD: an early form called acute GVHD that occurs soon after the transplant when white cells are on the rise, and a late form called chronic GVHD.

As used herein, the term “agonist” refers to an agent that increases the level and/or activity of the target, e.g., of NRP-2. As used herein, the term “agonist” refers to an agent which increases the expression and/or activity of the target by at least 10% or more, e.g. by 10% or more, 50% or more, 100% or more, 200% or more, 500% or more, or 1000% or more. The mutant semaphorin polypeptides as described herein are typically considered agonists herein.

As used herein, the term “Sema3F” refers to a member of the class III semaphorins that preferentially binds to NRP-2 as compared to NRP-1. Sequences for Sema3F polypeptides and nucleic acids for a number of species are known in the art, e.g. human Sema3F (NCBI Gene ID: 6405) polypeptide (SEQ ID NO: 1; NCBI Ref Seq: NP_004177) and nucleic acid (SEQ ID NO: 2; NCBI Ref Seq: NM_004186). The level of Sema3F can be assessed in blood, serum and/or plasma and the activity of Sema3F can be measured, e.g. by determining the level of binding of Sema3F to NRP-2, a select NRP-2 signaling response, changes in the activity of, and/or the level of an immune responsiveness parameter wherein increased Sema3F activity is evidenced by a reduced immune response and/or alloimmune response (e.g. cytokine responsiveness, priming, or cell migration following transplantation).

As used herein, “NRP-2” or “neuropilin-2” refers to a transmembrane glycoprotein receptor which recognizes class 3 semaphorins. NRPs regulate axon growth and angiogenesis. NRP2 can be distinguished from NRP1 in that NRP2 has a higher affinity for Sema-3F rather than Sema-3A. The sequences of NRP-2 genes, transcripts, and polypeptides are known in a variety of species, e.g. human NRP-2 mRNA (e.g. SEQ ID NO: 3; NCBI Ref Seq: NM_201266) and polypeptide (e.g. SEQ ID NO: 4; NCBI Ref Seq: NP_957718) sequences (NCBI Gene ID: 8828). NRP-2 comprises the A1 domain (e.g. the amino acids corresponding to positions 28-141 of SEQ ID NO: 4), the A2 domain (e.g. the amino acids corresponding to positions 149-265 of SEQ ID NO: 4), the B1 domain (e.g. the amino acids corresponding to positions 277-427 of SEQ ID NO: 4), and the B2 domain (e.g., the amino acids corresponding to positions 433-592 of SEQ ID NO: 4). Further discussion of NRP-2 structure can be found in the art, e.g., in Appleton et al. The EMBO Journal 2007 26:4901-4912; which is incorporated by reference herein in its entirety.

As used herein, the term “retains the ability to bind NRP-2” can refer to the ability of the mutant semaphorin polypeptide to bind to the binding site for wild-type semaphorin 3 polypeptides on NRP-2 in a manner such that the NRP-2 receptor is activated. It is expected that mutant semaphorin polypeptides comprising a mutation(s) confined to the proproteinase cleavage motif will retain the same or substantially similar binding properties with respect to neuropilin 2 as the wild-type semaphorin. Wild-type class 3 semaphorins bind to the CUB domain (two repeats of complement), and the FV/FVIII domain (two repeats of coagulation factor) on NRP-2 (see e.g., Nakamura and Goshima. Madame Curie Bioscience Database “Structural and Functional Relation of Neuropilins” Austin (TX): Landes Bioscience 2000-2013). Thus, at a minimum, a mutant semaphorin polypeptide as described herein will retain binding to the CUB and/or the FV/FVIII domain(s) of the neuropilin 2 receptor. Exemplary methods for detecting binding of a mutant semaphorin polypeptide to NRP-2 are summarized in e.g., Sharma et al. Front Cell Neurosci (2012) 6:28. Such exemplary methods include attraction/repulsion assays, neurite growth cone collapse assays, assays that measure staining of semaphorin binding to ectopically expressed NRP-2 (or the CUB and/or FV/FVIII domains) in COS or HEK-293 cells, co-immunoprecipitation assays or standard ligand binding assays. In one embodiment, the binding of a mutant semaphorin polypeptide to NRP-2 is assessed using a neurite growth cone collapse assay.

In another embodiment, “retains the ability to bind NRP-2” refers to a mutant semaphorin polypeptide having similar NRP-2 binding properties (e.g., dissociation constant K_{D}) relative to the wild-type semaphorin polypeptide. Previous studies have reported the K_{D} for binding of SEMA 3F to NRP2 to be ˜3.9±1.5 nM (see e.g., Geretti et al. J Biol Chem 282 (35):25698-25707 (2007) for exemplary methods to determine K_{D} of a mutant semaphorin polypeptide). Mutant semaphorin polypeptides having a K_{D} within this range (i.e., 2.4 to 5.4 nM) are preferred, however mutant semaphorin polypeptides retaining binding to NRP-2 with a K_{D} between 1.5-2.5 or 5.5-6.5 nM can still be useful provided that the NRP-2 receptor is activated. As will be understood by those of skill in the art, a functional method to assess binding of a mutant semaphorin polypeptide to NRP-2 is to measure the expression and/or activity of effectors downstream of NRP-2 (e.g., Ras/Rac second messenger system; LIMK 1, cofilin, Cdk5, CRAM, CRMP2, among others). In one embodiment, in order to retain the ability to bind NRP-2, mutations inside the sema domain (see e.g., Gherardi et al. Curr Opin Struct Biol 14(6):669-678 (2004)) of the wild-type semaphorin polypeptide should be limited to conservative amino acid substitutions only.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased,” “increase” or “enhance” or “activate” are all used herein to generally mean an increase of a property, level, or other parameter by a statistically significant amount; for the avoidance of any doubt, the terms “increased”N “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

The term “pharmaceutically acceptable” can refer to compounds and compositions which can be administered to a subject (e.g., a mammal or a human) without undue toxicity.

As used herein, the term “pharmaceutically acceptable carrier” can include any material or substance that, when combined with an active ingredient, allows the ingredient to retain biological activity and is non-reactive with the subject's immune system. Examples include, but are not limited to, any of the standard pharmaceutical carriers such as a phosphate buffered saline solution, emulsions such as oil/water emulsion, and various types of wetting agents. The term “pharmaceutically acceptable carriers” excludes tissue culture and bacterial culture media.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of, e.g., allograft rejection. A subject can be male or female.

A subject can be one who has been previously diagnosed with or identified as suffering from or having a condition in need of treatment (e.g. a subject undergoing an allograft or having an autoimmune disease) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

A “cancer cell” is a cancerous, pre-cancerous, or transformed cell, either in vivo, ex vivo, or in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is associated with, e.g., morphological changes, immortalization of cells, aberrant growth control, foci formation, anchorage independence, malignancy, loss of contact inhibition and density limitation of growth, growth factor or serum independence, tumor specific markers, invasiveness or metastasis, and tumor growth in suitable animal hosts such as nude mice. See, e.g., Freshney, CULTURE ANIMAL CELLS: MANUAL BASIC TECH. (3rd ed., 1994). As used herein, the term “cancer” refers to an uncontrolled growth of cells that interferes with the normal functioning of the bodily organs and systems. A subject who has a cancer or a tumor is a subject having objectively measurable cancer cells present in the subject's body. Included in this definition are benign and malignant cancers, as well as dormant tumors or micrometastases. Cancers that migrate from their original location and seed vital organs can eventually lead to the death of the subject through the functional deterioration of the affected organs.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment.

The term “consisting of” refers to compositions, methods, and respective components thereof as described herein, which are exclusive of any element not recited in that description of the embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), , Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (4 ed.), Cold Spring Harbor Laboratory Press, Cold Spring Harbor, N.Y., USA (2012); Davis et al., Basic Methods in Molecular Biology, Elsevier Science Publishing, Inc., New York, USA (1995); or Methods in Enzymology: Guide to Molecular Cloning Techniques Vol. 152, S. L. Berger and A. R. Kimmel Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments can perform functions in a different order, or functions can be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiment, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1) A semaphorin (SEMA) polypeptide, wherein the proproteinase cleavage motif is inactivated by mutation, and wherein the proproteinase cleavage motif is selected from the group consisting of: RXXR, RXKR, RXRR, RRXRR (SEQ ID NO: 15), and RRXKR (SEQ ID NO: 16).
2) The SEMA polypeptide of paragraph 1, wherein the proproteinase cleavage motif is selected from the group consisting of: RSRR (SEQ ID NO: 14), RRSRR (SEQ ID NO: 8), RTRR (SEQ ID NO: 13), RRTRR (SEQ ID NO: 11), RFRR (SEQ ID NO: 12), and RRFRR (SEQ ID NO: 10).
3) The SEMA polypeptide of paragraph 1, wherein its wild-type SEMA polypeptide binds to neuropilin 2, and wherein the mutant SEMA polypeptide retains the ability to bind to neuropilin 2.
4) The SEMA polypeptide of paragraph 1, wherein the SEMA polypeptide is a human SEMA polypeptide.
5) The SEMA polypeptide of paragraph 1, wherein the SEMA polypeptide is selected from a SEMA 3A polypeptide, SEMA 3C polypeptide, a SEMA 3F polypeptide and a SEMA 3G polypeptide.
6) The SEMA polypeptide of paragraph 2, wherein the proproteinase cleavage motif comprises amino acids:
   (i) 582-586 of SEQ ID NO: 1,
   (ii) 583-586 of SEQ ID NO: 1,
   (iii) 550-555 of SEQ ID NO: 5,
   (iv) 551-555 of SEQ ID NO: 5,
   (v) 548-552 of SEQ ID NO: 6,
   (vi) 549-552 of SEQ ID NO: 6,
   (vii) 557-561 of SEQ ID NO: 7, or
   (viii) 558-561 of SEQ ID NO:7.
7) The SEMA polypeptide of paragraph 2, wherein the proproteinase cleavage motif RRSRR (SEQ ID NO: 8) is inactivated by mutating the second and fourth arginines in the motif.
8) The SEMA polypeptide of paragraph 2, wherein the proproteinase cleavage motif RRSRR (SEQ ID NO: 8) is inactivated by mutating arginine 583 and arginine 586 of SEQ ID NO: 1 to alanine.
9) A nucleic acid molecule encoding any one of the SEMA polypeptides of paragraphs 1-8.
10) The nucleic acid molecule of paragraph 9, wherein the nucleic acid molecule is a cDNA or a modified RNA.
11) A vector comprising the nucleic acid molecule of paragraph 9.
12) The vector of paragraph 11, wherein the vector is a viral vector.
13) The vector of paragraph 12, wherein the viral vector is an adenoviral vector or an adeno-associated viral (AAV) vector.
14) A cell comprising the nucleic acid of any one of paragraphs 9-10, or the vector of any one of paragraphs 11-13.
15) A pharmaceutical composition comprising the SEMA polypeptide of any one of paragraphs 1-8, the nucleic acid of any one of paragraphs 9-10, the vector of any one of paragraphs 11-13 , or the cell of paragraph 14.
16) A method of inhibiting transplant or allograft rejection in a subject in need thereof, comprising administering to the subject a therapeutically effective amount of a pharmaceutical composition of paragraph 15.
17) A method of inhibiting transplant or allograft rejection comprising contacting transplant tissue with an amount of a pharmaceutical composition of paragraph 15 that is effective to suppress the immune system of the subject.
18) A method of suppressing the immune system in a subject in need thereof comprising administering to the subject a pharmaceutical composition of paragraph 15.
19) A method of treating an inflammatory condition in a subject in need thereof comprising administering to the subject a pharmaceutical composition of paragraph 15.
20) The method of paragraph 19, wherein the inflammatory condition is an autoimmune disease.
21) The method of paragraph 17, wherein the transplant tissue is contacted in vivo prior to removal from a tissue donor.
22) The method of paragraph 17, wherein the transplant tissue is contacted ex vivo or in vitro.
23) A multispecific agent comprising a semaphorin polypeptide that binds to neuropilin 2, and an agent that binds an immunomodulator polypeptide.
24) The multispecific agent of paragraph 23, wherein the semaphorin polypeptide comprises a semaphorin family immunoglobulin domain.
25) The multispecific agent of paragraph 24, wherein the semaphorin family immunoglobulin domain is a SEMA3F immunoglobulin domain.
26) The multispecific agent of paragraph 23, wherein the semaphorin polypeptide is selected from semaphorin 3F, semaphorin 3G, semaphorin 3A, and semaphorin 3C.
27) The multispecific agent of any one of paragraphs 23-26, wherein the immunomodulator polypeptide is an immune checkpoint polypeptide.
28) The multispecific agent of paragraph 27, wherein the immune checkpoint polypeptide is selected from PD-L1, TIM-1, TIM-3, PD-1, CTLA4, TIGIT, LAG3, VISTA, 4-1BBL, B7-H3 and B7-DC.
29) The multispecific agent of any one of paragraphs 27 and 28, wherein the agent that binds an immunomodulator polypeptide inhibits the immune checkpoint polypeptide.
30) The multispecific agent of paragraph 23, wherein the semaphorin polypeptide has a mutation that inactivates the proproteinase cleavage site RRSRR (SEQ ID NO: 8).
31) A composition comprising a first semaphorin polypeptide and a second semaphorin polypeptide, joined by a linker.
32) The composition of paragraph 31, wherein the first semaphorin polypeptide and the second semaphorin polypeptide are the same.
33) A method of treating cancer, the method comprising administering to a subject in need thereof a composition comprising a SEMA polypeptide of any one of paragraphs 1-8.
34) The method of paragraph 33, further comprising administering an immune checkpoint inhibitor.
35) The method of paragraph 34, wherein the immune checkpoint inhibitor inhibits a checkpoint molecule selected from the group consisting of PD-L1, TIM-1, TIM-3, PD-1, CTLA4, TIGIT, LAG3, VISTA, 4-1BBL, B7-H3 and B7-DC.
36) The method of any one of paragraphs 33-35, further comprising administering an inhibitor of neuropilin 2.
37) The method of any one of paragraphs 33-36, further comprising administering a chemotherapeutic or anti-cancer agent, or radiation treatment.
38) The method of any one of paragraphs 33-37, wherein the cancer expresses neuropilin 2.
39) A method of treating cancer, the method comprising administering to a subject in need thereof a pharmaceutical composition of paragraph 15.
40) The method of paragraph 39, further comprising administering an immune checkpoint inhibitor.
41) The method of paragraph 40, wherein the immune checkpoint inhibitor inhibits a checkpoint molecule selected from the group consisting of PD-L1, TIM-1, TIM-3, PD-1, CTLA4, TIGIT, LAG3, VISTA, 4-1BBL, B7-H3 and B7-DC.
42) The method of any one of paragraphs 39-41, further comprising administering an inhibitor of neuropilin 2.
43) The method of any one of paragraphs 39-42, further comprising administering a chemotherapeutic or anti-cancer agent, or radiation treatment.
44) The method of any one of paragraphs 39-43, wherein the cancer expresses neuropilin 2.
45) A method of treating cancer, the method comprising administering to a subject in need thereof a multispecific agent of any one of paragraphs 23-29.
46) The method of paragraph 45, further comprising administering an immune checkpoint inhibitor.
47) The method of paragraph 46, wherein the immune checkpoint inhibitor inhibits a checkpoint molecule selected from the group consisting of PD-L1, TIM-1, TIM-3, PD-1, CTLA4, TIGIT, LAG3, VISTA, 4-1BBL, B7-H3 and B7-DC.
48) The method of any one of paragraphs 45-47, further comprising administering an inhibitor of neuropilin 2.
49) The method of any one of paragraphs 45-48, further comprising administering a chemotherapeutic or anti-cancer agent, or radiation treatment.
50) The method of any one of paragraphs 45-49, wherein the cancer expresses neuropilin 2.
51) A method of inhibiting metastasis of a cancer, the method comprising administering to a subject in need thereof a composition comprising a SEMA polypeptide of any one of paragraphs 1-8.
52) The method of paragraph 51, wherein the cancer expresses neuropilin 2.
53) A method of inhibiting metastasis of a cancer that expresses neuropilin 2, the method comprising administering to a subject in need thereof a pharmaceutical composition of paragraph 15.
54) The method of paragraph 53, wherein the cancer expresses neuropilin 2.
55) A method of inhibiting metastasis of a cancer that expresses neuropilin 2, the method comprising administering to a subject in need thereof a multispecific agent of any one of paragraphs 23-29.
56) The method of paragraph 55, wherein the cancer expresses neuropilin 2.

### EXAMPLES

The following provides non-limiting Examples demonstrating and supporting the technology as described herein.

### Example 1

Neuropilin-2 is expressed on human T cells and T cell lines (Jurkat T cells) and the binding of Sema3F results in an activation response. A mutant semaphorin polypeptide as described herein retains the binding of NRP-2, resulting in an activation response.

### Example 2

The treatment of allograft recipients with a composition expressing a mutant semaphorin polypeptide will prolong survival.

The injection i.p of cells overexpressing a mutant semaphorin polypeptide into mice recipients of cardiac transplants will be associated with a prolongation of allograft survival, and a delay in the acute rejection response. The prolongation of allograft survival induced by the mutant semaphorin polypeptide will be increased compared to the prolongation of allograft survival elicited by a comparable dose of SEMA3F polypeptide. It is contemplated herein that the time period between doses of the mutant semaphorin polypeptide is increased by at least 6 h, at least 12 h, at least 15 hours, at least 24 h, at least 36 h, at least 48 h, or at least 72 h or more, compared to the time period between doses of a comparable dose of SEMA3F polypeptide. As will be appreciated by one of skill in the art, the timing between doses is determined by the half-life of the polypeptide, the bioavailability of the polypeptide, the threshold level of the polypeptide needed for a given response, and the therapeutic window. One of skill in the art can determine appropriate timing for a given composition comprising a mutant semaphorin polypeptide as described herein.

It is specifically contemplated herein that:
(i) A mutant semaphorin polypeptide or composition thereof can be utilized as an anti-inflammatory or immunomodulator agent in many inflammatory disease states, and
(ii) A mutant semaphorin polypeptide or composition thereof can be utilized in treating and/or preventing allograft rejection. Augmenting interactions that activate NRP2 can serve as an immunosuppressant.

### Example 3

A mutant semaphorin polypeptide acts as an Immunosuppressant in vivo to inhibit Acute Allograft Rejection

Balb/C donor hearts are transplanted into C56BL6 mice. Control mice will experience rejection on day 7-8. IV injection of an adenovirus encoding a mutant semaphorin polypeptide as described gereub into mice following cardiac transplantation will prolong survival, for example, up to day 40 or beyond.

Rapamycin at 0.2 mg/kg is administered on day 0-2 and the mutant semaphorin polypeptide is administered. No additive graft prolongation effect is expected in this limited model (no significant prolongation of survival).

CD4+ T cells are obtained and sorted into CD25^{neg} T effector subsets from WT, NRP-2+/− (Hets) and NRP-2−/− (KO) mice on a C57BL/6 background. Mitogen-induced proliferation and cytokine production (ELISPOT) are assessed. Markedly enhanced activation responses will be observed in whole populations of CD4+ T cells as well as CD25^{neg} subsets derived from NRP-2 Hets and NRP-2 KO mice. Sorted populations of CD4+ CD25^{neg} T effector subsets are also cultured with increasing concentrations of mitogen (anti-CD3) in the presence of anti-CD28. CD4+ T cells will proliferate maximally in response to costimulatory signals, however, NRP-2 KO cells will likely remain hyperactive and produce significantly more IFNγ and IL-2 than CD4+ T cells derived from WT mice.

### Chronic Rejection

Minor MHC mismatched B6.C-H2^{bm12} (BM12) allografts are transplanted into C57BL/6 (wild type/WT), NRP-2+/− (Het on BL6) or NRP-2−/− (KO on BL6) mice. It is expected that allografts in WT recipients will survive long term but develop chronic rejection after ˜30 days post transplantation with marked evidence of disease present by day 45. Long-term survival in this model is reported to be associated with the expansion of T regulatory cells by day 21 post transplantation, that limit the expansion of T effectors. Survival will likely be reduced in NRP-2+/− Het recipients and significantly reduced in NRP-2−/− KO recipients (P<0.05).

### Sema3F Modulates PI-3K/Akt-mTOR Signaling

U87MG cells, known to express high levels of NRP-2, are treated with a mutant semaphorin polypeptide at a level known to stimulate a signaling response (˜640 ng/mL). Inhibition of pAkt (mTORC2) and pS6K (mTORC1) dependent activation is expected. Peak effects of the mutant semaphorin polypeptide are expected at ˜600 ng/ml and the concentration that produces a peak effect of the mutant semaphorin polypeptide will used for all signaling analyses.

In addition, NRP-2-expressing Jurkat T cells can be treated with increasing concentrations of the mutant semaphorin polypeptide or composition thereof for 30 min, and expression of pAkt(S473) was evaluated by Western blot. Expression is expected to be reduced following treatment with high concentrations of the mutant semaphorin polypeptide.

### Example 4

Mice are injected with control adenovirus or adenovirus encoding a mutant semaphorin polypeptide as described above herein. At day 3 and day 5 after adenovirus injection, the mice are further treated with oxozalone to induce ear swelling. Mice receiving the mutant semaphorin polypeptide treatment are expected to demonstrate reduced swelling relative to the mice receiving the control treatments.

### Example 5

The immunoregulatory function of a mutant semaphorin polypeptide is evaluated by examining the Treg phenotype at early times post-transplant, e.g., on day 5. It is expected that no differences will be observed in CD3, CD4, CD8 and Tregs, as determined using FACS.

### Example 6

To study the effect of the mutant semaphorin polypeptide in vivo, an adenovirus containing a nucleic acid encoding a mutant semaphorin polypeptide or an empty control is administered into mice in a heart transplant model. It is expected that the vector will permit measurable expression of the mutant semaphorin polypeptide in e.g., the liver. It is expected that the peak expression of the mutant semaphorin polypeptide will occur at approximately day 14 following administration.

### Example 7

The growth inhibitory activity of Sema3F and a Sema3F mutant as described herein was examined using human endothelial cells in culture. Increasing doses of non-mutant (“Sema3F”) and mutant (“furin-uncleavable”) Sema3F as described herein were added to HUVEC cultures with, or without heparin, and cell numbers monitored.

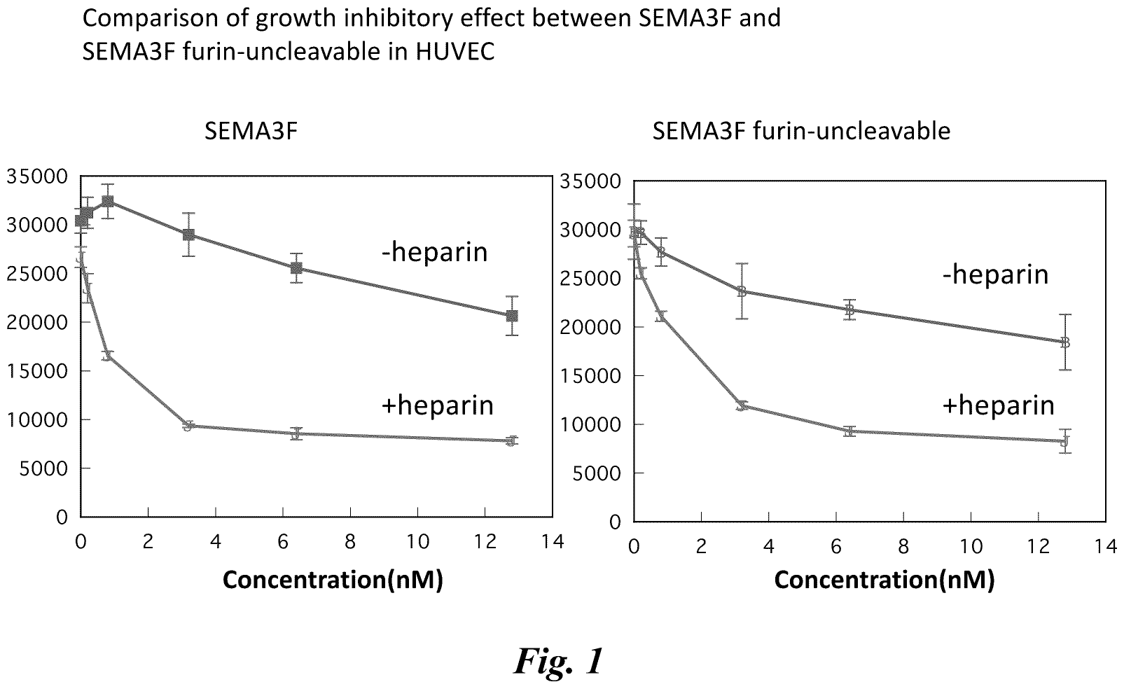

Both non-mutant Sema3F (left panel) and mutant Sema3f (right panel) as described herein were effective in a dose-dependent manner to inhibit growth of the endothelial cells, thereby indicating that the mutant retains growth inhibitory activity of the non-mutant (FIG. 1). The activity of both mutant and non-mutant Sema3F was enhanced by heparin, which is known to enhance the binding of semaphorin polypeptides binding to their receptors.

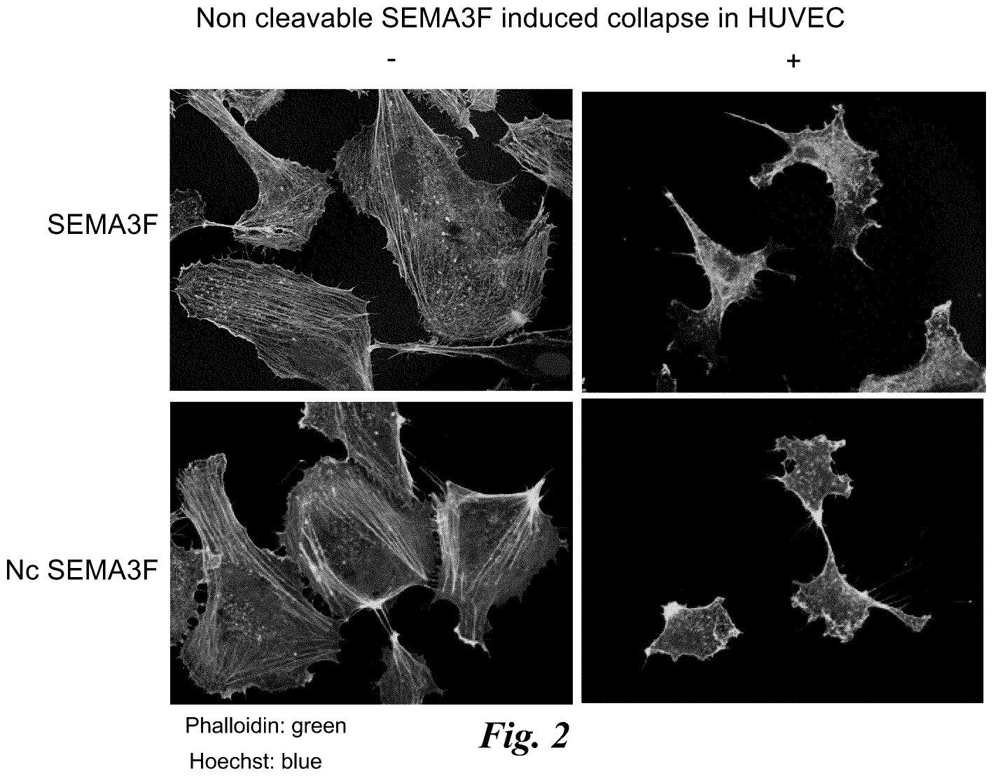

HUVEC cultures treated with non-mutant and mutant Sema3F were examined by staining f-actin filaments with phalloidin. Both non-mutant, or cleavable, Sema 3F (top) and mutant, non-cleavable (Nc) Sema3F (bottom) induced f-actin filament collapse (FIG. 2), which is consistent with the mutant acting by the same pathway as the non-mutant in its effects upon endothelial cell proliferation.

### Example 8

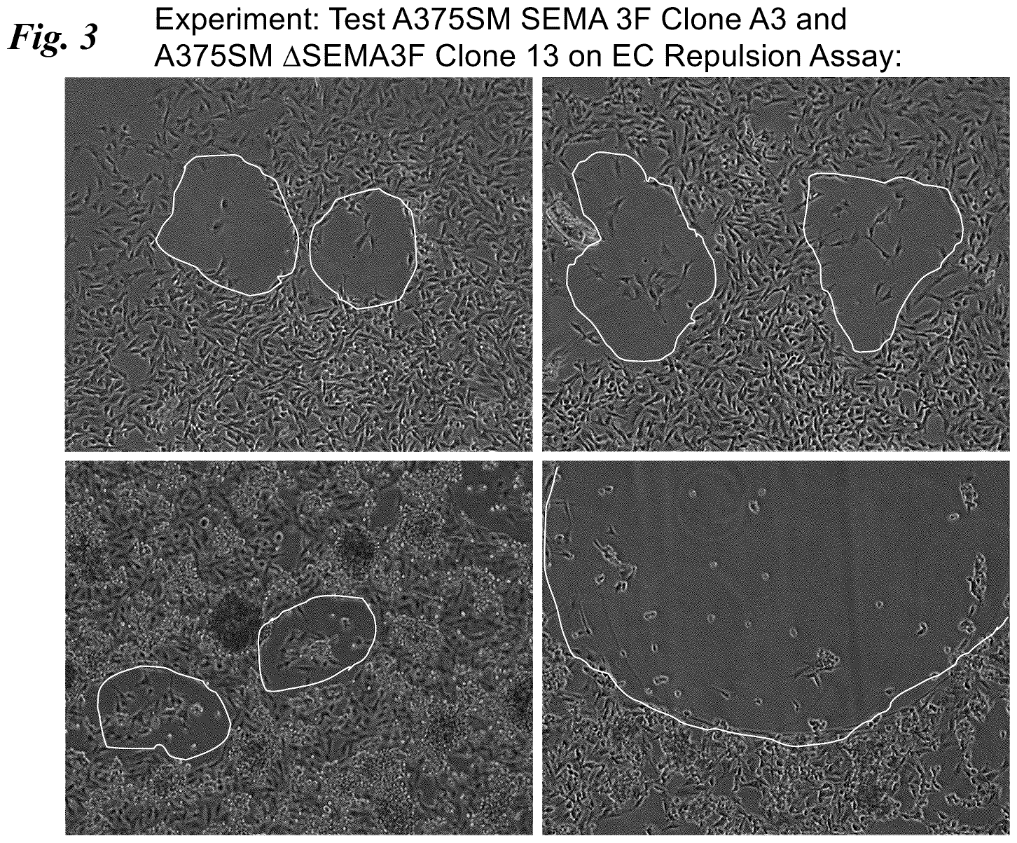

Sema3F has potent anti-angiogenesis effects, including chemorepulsive activity on vascular and lymphatic endothelial cells. Mutant Sema3F as described herein was tested for its effect on cultured endothelial cells in the manner described, for example, in Bielenberg et al., J. Clin. Invest. 114: 1260-1271. FIG. 3 shows the results of a chemorepulsion assay using cells expressing wild-type (top panels) and mutant (bottom panels) Sema3F. The data show that endothelial cell migration is halted and even regresses to a similar extent in the presence of either wild-type or mutant Sema3F.

### Example 9

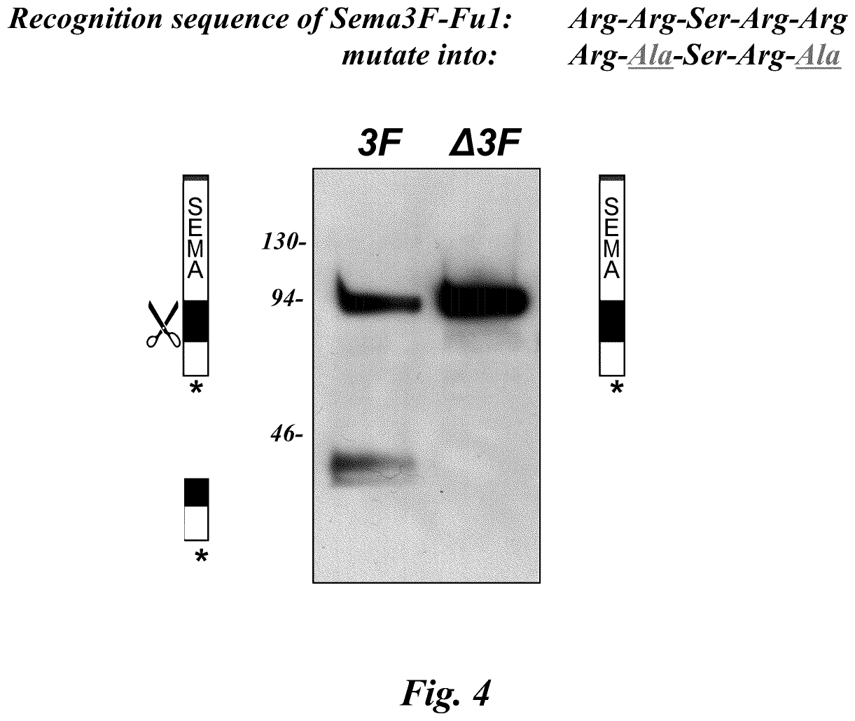

The wildtype human Semaphorin-3F (SEMA3F) protein has a furin proproteinase cleavage site: Arg-Arg-Ser-Arg-Arg (RRSRR; SEQ ID NO: 8). A mutant Semaphorin-3F protein was engineered with an altered sequence encoding: Arg-Ala-Ser-Arg-Ala (RASRA; SEQ ID NO: 9), which is not recognized by furin and therefore not cleaved (FIG. 4). Human melanoma cells A375SM were transfected with the wildtype SEMA3F expression (exons only) gene construct or mutated SEMA3F gene construct following a CMV promoter and containing a C-terminal myc tag for detection. After transfection, serum-free conditioned medium was collected from the cells and separated by SDS-PAGE and immunoblotted with anti-mye antibodies. In FIG. 4 (left lane), cells transfected with wild type SEMA3F secreted myc-tagged proteins of 95kDa and 30kDa, while in the

FIG. 4 (right lane) cells transfected with mutated SEMA3F secreted only the full-length, uncleaved 95kDa myc-tagged SEMA3F protein.

SEQ ID NO: 2

| | |
|---|---|
| gagcgcccct gagccttccc atggcccggg ctggggcccg ggccctcggc tgctgacgcg | 60 |
| cccgaagccc gcggaaccgg ttaagccgcg gccgcggcgc cgatcccggc tgaggcgcag | 120 |
| cggcgagagg tcgcgggcag ggccatggcc ccggggggcc gctagcgcgg accggcccaa | 180 |
| cgggagccgc tccgtgccgc cgccgccgcc cgggcgccca ggccccgccg ctgcggaaga | 240 |
| ggtttctaga gagtggagcc tgcttcctgg gccctaggcc cctcccacaa tgcttgtcgc | 300 |
| cggtcttctt ctctgggctt ccctactgac cggggcctgg ccatccttcc ccacccagga | 360 |
| ccacctcccg gccacgcccc gggtccggct ctcattcaaa gagctgaagg ccacaggcac | 420 |
| cgcccacttc ttcaacttcc tgctcaacac aaccgactac cgaatcttgc tcaaggacga | 480 |
| ggaccacgac cgcatgtacg tgggcagcaa ggactacgtg ctgtccctgg acctgcacga | 540 |
| catcaaccgc gagcccctca ttatacactg ggcagcctcc ccacagcgca tcgaggaatg | 600 |
| cgtgctctca ggcaaggatg tcaacggcga gtgtgggaac ttcgtcaggc tcatccagcc | 660 |
| ctggaaccga acacacctgt atgtgtgcgg gacaggtgcc tacaacccca tgtgcaccta | 720 |
| tgtgaaccgc ggacgccgcg cccaggccac accatggacc cagactcagg cggtcagagg | 780 |
| ccgcggcagc agagccacgg atggtgccct ccgcccgatg cccacagccc cacgccagga | 840 |
| ttacatcttc tacctggagc ctgagcgact cgagtcaggg aagggcaagt gtccgtacga | 900 |
| tcccaagctg gacacagcat cggccctcat caatgaggag ctctatgctg gtgtgtacat | 960 |
| cgattttatg ggcactgatg cagccatctt ccgcacactt ggaaagcaga cagccatgcg | 1020 |
| cacggatcag tacaactccc ggtggctgaa cgacccgtcg ttcatccatg ctgagctcat | 1080 |
| tcctgacagt gcggagcgca atgatgataa gctttacttc ttcttccgtg agcggtcggc | 1140 |
| agaggcgccg cagagccccg cggtgtacgc ccgcatcggg cgcatttgcc tgaacgatga | 1200 |
| cggtggtcac tgttgcctgg tcaacaagtg gagcacattc ctgaaggcgc ggctcgtctg | 1260 |
| ctctgtcccg ggcgaggatg gcattgagac tcactttgat gagctccagg acgtgtttgt | 1320 |
| ccagcagacc caggacgtga ggaaccctgt catttacgct gtctttacct cctctggctc | 1380 |
| cgtgttccga ggctctgccg tgtgtgtcta ctccatggct gatattcgca tggtcttcaa | 1440 |
| cgggcccttt gcccacaaag aggggcccaa ctaccagtgg atgcccttct cagggaagat | 1500 |
| gccctaccca cggccgggca cgtgccctgg tggaaccttc acgccatcta tgaagtccac | 1560 |
| caaggattat cctgatgagg tgatcaactt catgcgcagc cacccactca tgtaccaggc | 1620 |
| cgtgtaccct ctgcagcggc ggcccctggt agtccgcaca ggtgctccct accgccttac | 1680 |
| cactattgcc gtggaccagg tggatgcagc cgacgggcgc tatgaggtgc ttttcctggg | 1740 |
| cacagaccgc gggacagtgc agaaggtcat tgtgctgccc aaggatgacc aggagttgga | 1800 |
| ggagctcatg ctggaggagg tggaggtctt caaggatcca gcacccgtca agaccatgac | 1860 |
| catctcttct aagaggcaac aactctacgt ggcgtcagcc gtgggtgtca cacacctgag | 1920 |
| cctgcaccgc tgccaggcgt atggggctgc ctgtgctgac tgctgccttg cccgggaccc | 1980 |
| ttactgtgcc tgggatggcc aggcctgctc ccgctataca gcatcctcca agaggcggag | 2040 |
| ccgccggcag gacgtccggc acggaaaccc catcaggcag tgccgtgggt tcaactccaa | 2100 |
| tgccaacaag aatgccgtgg agtctgtgca gtatggcgtg gccggcagcg cagccttcct | 2160 |
| tgagtgccag ccccgctcgc cccaagccac tgttaagtgg ctgttccagc gagatcctgg | 2220 |
| tgaccggcgc cgagagattc gtgcagagga ccgcttcctg cgcacagagc agggcttgtt | 2280 |
| gctccgtgca ctgcagctca gcgatcgtgg cctctactcc tgcacagcca ctgagaacaa | 2340 |
| ctttaagcac gtcgtcacac gagtgcagct gcatgtactg ggccgggacg ccgtccatgc | 2400 |
| tgccctcttc ccaccactgt ccatgagcgc cccgccaccc ccaggcgcag gccccccaac | 2460 |
| gcctccttac caggagttag cccagctgct ggcccagcca gaagtgggcc tcatccacca | 2520 |
| gtactgccag ggttactggc gccatgtgcc ccccagcccc agggaggctc caggggcacc | 2580 |
| ccggtctcct gagccccagg accagaaaaa gccccggaac cgccggcacc accctccgga | 2640 |
| cacatgaggc cagctgcctg tgcctgccat gggccagcct agcccttgtc ccttttaata | 2700 |
| taaaagatat atatatatat atatatatat aaaatatcta tattctatac acaccctgcc | 2760 |
| cctgcaaaga cagtatttat tggtgggttg aatatagcct gcctcagtgg cagcatcct | 2820 |
| caaaacttag acccatgctg gtcagagacg gcagaaaaca gagcctgcct aaccaggccc | 2880 |
| agccagttgg tggggccagg ccaggaccac acagtcccca gactcagctg gaagtctacc | 2940 |
| tgctggacag cctccgccaa gatctacagg acaaagggag ggagcaagcc ctactcggat | 3000 |
| ggggcacgga ctgtccacct tttctgatgt gtgttgtcag cctgtgctgt ggcatagaca | 3060 |
| tggatgcgag gaccactttg gagactgggg tggcctcaag agcacacaga gaagggaaga | 3120 |
| aggggccatc acaggatgcc agcccctgcc tgggttgggg gcactcagcc acgaccagcc | 3180 |
| ccttcctggg tatttattct ctatttattg gggataggag aagaggcatc ctgcctgggt | 3240 |
| gggacagcct cttcagcccc ttctcccctc cccgcctggc cagggcaggg ccaccccact | 3300 |
| ctacctcctt agctttccct gtgccacttt gactcagagg ctgggagcat agcagagggg | 3360 |
| ccaggcccag gcagagctga cgggaggccc cagctctgag gggagggggt ccgtggtaga | 3420 |
| ggcctggggc cggtagaggc tccccagggc tcccttatgt ccaccacttc aggggatggg | 3480 |
| tgtggatgta attagctctg gggggcagtt gggtagatgg gtgggggctc ctggtggcct | 3540 |
| tctgctgccc aggccacagc cgcctttggg ttccatcttg ctaataaaca ctggctctgg | 3600 |
| gactaga | 3607 |

SEQ ID NO: 3

| | |
|---|---|
| agagatcgcg agcgaggcac cagcctgcag ccggccccca gcacatcctc agccgcacag | 60 |
| acactcggcg aggtggaggt gagggcgggc gccagcgaac tcggagaggg gctcgctcac | 120 |
| tcccaggcga tcccagccgc caccgccgcc gcaccagcag cagcaacagc agcagcagct | 180 |
| tccttcctca gactcccctc gagaggctgg ccaagcgggt gtagccgttg ggggaggctc | 240 |
| ccgccggggg aacccggcga ggacaagagc agggcggccg ccttccactc gggctgtccg | 300 |
| gcggcggctg cctccgcccg tgtgtccgtc aagggtgccg cgggatgtgt gtcagtttac | 360 |
| gcctctgaga tcacacagct gcctgggggc cgtgtgatgc ccaaggcaag tcttggtttt | 420 |
| aattattatt attatcatta ttgttacgct tggctttcgg gaaatactcg tgatatttgt | 480 |
| aggataaagg aaatgacact ttgaggaact ggagagaaca tatatgcgtt ttgtttttaa | 540 |
| gaggaaaacc gtgttctctt cccggcttgt tccctctttg ctgatttcag gagctactct | 600 |
| cctcctggtg aggtggaaat tccagcaaga atagaggtga agacaagcca ccaggactca | 660 |
| ggagggaaac gctgaccatt agaaacctct gcataagacg ttgtaaggag gaaaataaaa | 720 |
| gagagaaaaa cacaaagatt taaacaagaa acctacgaac ccagctctgg aaagagccac | 780 |
| cttctccaaa atggatatgt ttcctctcac ctgggttttc ttagccctct acttttcaag | 840 |
| acaccaagtg agaggccaac cagacccacc gtgcggaggt cgtttgaatt ccaaagatgc | 900 |
| tggctatatc acctctcccg gttaccccca ggactacccc tcccaccaga actgcgagtg | 960 |
| gattgtttac gcccccgaac ccaaccagaa gattgtcctc aacttcaacc ctcactttga | 1020 |
| aatcgagaag cacgactgca agtatgactt tatcgagatt cgggatgggg acagtgaatc | 1080 |
| cgcagacctc ctgggcaaac actgtgggaa catcgccccg cccaccatca tctcctcggg | 1140 |
| ctccatgctc tacatcaagt tcacctccga ctacgcccgg cagggggcag gcttctctct | 1200 |
| gcgctacgag atcttcaaga caggctctga agattgctca aaaaacttca caagccccaa | 1260 |
| cgggaccatc gaatctcctg ggtttcctga gaagtatcca cacaacttgg actgcacctt | 1320 |
| taccatcctg gccaaaccca agatggagat catcctgcag ttcctgatct ttgacctgga | 1380 |
| gcatgaccct ttgcaggtgg gagaggggga ctgcaagtac gattggctgg acatctggga | 1440 |
| tggcattcca catgttggcc ccctgattgg caagtactgt gggaccaaaa caccctctga | 1500 |
| acttcgttca tcgacgggga tcctctccct gacctttcac acggacatgg cggtggccaa | 1560 |
| ggatggcttc tctgcgcgtt actacctggt ccaccaagag ccactagaga actttcagtg | 1620 |
| caatgttcct ctgggcatgg agtctggccg gattgctaat gaacagatca gtgcctcatc | 1680 |
| tacctactct gatgggaggt ggacccctca acaaagccgg ctccatggtg atgacaatgg | 1740 |
| ctggaccccc aacttggatt ccaacaagga gtatctccag gtggacctgc gctttttaac | 1800 |
| catgctcacg gccatcgcaa cacagggagc gatttccagg gaaacacaga atggctacta | 1860 |
| tgtcaaatcc tacaagctgg aagtcagcac taatggagag gactggatgg tgtaccggca | 1920 |
| tggcaaaaac cacaaggtat ttcaagccaa caacgatgca actgaggtgg ttctgaacaa | 1980 |
| gctccacgct ccactgctga caaggtttgt tagaatccgc cctcagacct ggcactcagg | 2040 |
| tatcgccctc cggctggagc tcttcggctg ccgggtcaca gatgctccct gctccaacat | 2100 |
| gctggggatg ctctcaggcc tcattgcaga ctcccagatc tccgcctctt ccacccagga | 2160 |
| atacctctgg agccccagtg cagcccgcct ggttagcagc cgctcgggct ggttccctcg | 2220 |
| aatccctcag gcccagcccg gtgaggagtg gcttcaggta gatctgggaa cacccaagac | 2280 |
| agtgaaaggt gtcatcatcc agggagcccg cggaggagac agtatcactg ctgtggaagc | 2340 |
| cagagcattt gtgcgcaagt tcaaagtctc ctacagccta aacggcaagg actgggaata | 2400 |
| cattcaggac cccaggaccc agcagccaaa gctgttcgaa gggaacatgc actatgacac | 2460 |
| ccctgacatc cgaaggtttg accccattcc ggcacagtat gtgcgggtat acccggagag | 2520 |
| gtggtcgccg gcggggattg ggatgcggct ggaggtgctg ggctgtgact ggacagactc | 2580 |
| caagcccacg gtagagacgc tgggacccac tgtgaagagc gaagagacaa ccacccccta | 2640 |
| ccccaccgaa gaggaggcca cagagtgtgg ggagaactgc agctttgagg atgacaaaga | 2700 |
| tttgcagctc ccttcgggat tcaattgcaa cttcgatttc ctcgaggagc cctgtggttg | 2760 |
| gatgtatgac catgccaagt ggctccggac cacctgggcc agcagctcca gcccaaacga | 2820 |
| ccggacgttt ccagatgaca ggaatttctt gcggctgcag agtgacagcc agagagaggg | 2880 |
| ccagtatgcc cggctcatca gcccccctgt ccacctgccc cgaagcccgg tgtgcatgga | 2940 |
| gttccagtac caggccacgg gcggccgcgg ggtggcgctg caggtggtgc gggaagccag | 3000 |
| ccaggagagc aagttgctgt gggtcatccg tgaggaccag ggcggcgagt ggaagcacgg | 3060 |
| gcggatcatc ctgcccagct acgacatgga gtaccagatt gtgttcgagg gagtgatagg | 3120 |
| gaaaggacgt tccggagaga ttgccattga tgacattcgg ataagcactg atgtcccact | 3180 |
| ggagaactgc atggaaccca tctcggcttt tgcaggtgag aattttaaag tggacatccc | 3240 |
| agaaatacat gagagagaag gatatgaaga tgaaattgat gatgaatacg aggtggactg | 3300 |
| gagcaattct tcttctgcaa cctcagggtc tggcgccccc tcgaccgaca aagaaaagag | 3360 |
| ctggctgtac accctggatc ccatcctcat caccatcatc gccatgagct cactgggcgt | 3420 |
| cctcctgggg gccacctgtg caggcctcct gctctactgc acctgttcct actcgggcct | 3480 |
| gagctcccga agctgcacca cactggagaa ctacaacttc gagctctacg atggccttaa | 3540 |
| gcacaaggtc aagatgaacc accaaaagtg ctgctccgag gcatgacgga ttgcacctga | 3600 |
| atcctatctg acgtttcatt ccagcaagag gggctgggga agattacatt tttttttcct | 3660 |
| ttggaaactg aatgccataa tctcgatcaa accgatccag aataccgaag gtatggacag | 3720 |
| gacagaaaag cgagtcgcag gaggaaggga gatgcagccg cacaggggat gattaccctc | 3780 |
| ctaggaccgc ggtggctaag tcattgcagg aacggggctg tgttctctgc tgggacaaaa | 3840 |
| caggagctca tctctttggg gtcacagttc tattttgttt gtgagtttgt attattatta | 3900 |
| ttattattat tattattata ttttatttct ttggtctgtg agcaactcaa agaggcagaa | 3960 |
| gaggagaatg acttttccag aatagaagtg gagcagtgat cattattctc cgctttctct | 4020 |
| ttctaatcaa cacttgaaaa gcaaagtgtc ttttcagcct ttccatcttt acaaataaaa | 4080 |
| ctcaaaaaag ccgtccagct tatcccatcc tctgattgtc ttctgactta agggatttac | 4140 |
| tgtggtgtag gttctgccag ccaaccctac aagctgccat ttccagtcct agcatttaag | 4200 |
| taggatgttg ttgcctttaa cttttcttat ccaggggaaa attgccattt tagggtcagc | 4260 |
| atgaacagct ctttcttgta tgcgatttaa aacaaactgg aaaggaaact tcacacgtca | 4320 |
| aaatccatag aagcgcctgg acgaggctta aagtgctttg tgagtgaata ggagccattc | 4380 |
| gctaattcta gacccacagt gtctggtggt ggggcttccc ttgtggggct tctggtggtg | 4440 |
| gttttgcctt ttcttttccc tcctccatgt tcttctaaaa catatacata tatacataca | 4500 |
| cacatacaca tattcttcag gtctctaagc ccctggaagc agcattgtgt gatattctca | 4560 |
| gaggcagggg aaaatagagg gaaaaataga gactattggt atgttctccc catcagcgag | 4620 |
| ttattgtaac tggtcaccac tggacgggaa ggagaacaga ggagagggaa agagaagccc | 4680 |
| aacctctgtg atcatatgag ggccaaggct gagcagtgta gacagagacc ctttgaaatg | 4740 |
| catttgtctc tcaaatagac tagtaaacac cgacttctcc tttgggttac aaacaccatt | 4800 |
| tcaacctttc gggagagtca gagctaggat gtacaagaac tgattctaac cagaagtccg | 4860 |
| caagtactgt ggacaagaat gcttaaccat gctgcttcag ccttgagaga cctaggttct | 4920 |
| tacacatatg cacacacgca tacacacatg cacgcacaca cacatacaca catgcacgca | 4980 |
| cgcacgcatg cacaccaatt tatgttttta ttaagtgcct tgaaaaaatg aagaaaaatg | 5040 |
| tattttccct ttatgtaaaa attagtgaat atcttatgaa ttaaggcatt cctctttccc | 5100 |
| taaccccgat ggctccattc ccaagtaccc caactcactg ctgatcctat taaaggaatg | 5160 |
| agtcctgcta cccgagtggt agtcatagcc ctagatgact ctcaactact cttcaaaggg | 5220 |
| aggcatcagg aatagaatga aactgtgtga aggataagat tgttcgcatc aagatccaaa | 5280 |
| tcttgatttc atattaacgc ctaaggattg cctgtgtgct ggaaatatat ttgaaactca | 5340 |
| accagtatgc ccagcctatt gcatatcatt gtcagaccat ttttgctgct gtggtcaccc | 5400 |
| acgatttcat ttgtcttata cccaggtgaa aggggaaggg tgaatgggac tggctggttc | 5460 |
| ctttaaatgt taacttatgg aaatgctagt tcaaatggta atgtcacagt gttttgtatg | 5520 |
| cagagagcaa gagttcaacc aacagctgtt tattcatgtg tgtgtgtctt tgctgctttg | 5580 |
| agttctctgt atctactgtg tatgtgaatg gtcatgtggg actcagtggt ggtgttgtga | 5640 |
| ctttgaccta gggtccgagt gtcacagctg atcttggcac tcggcactca ttggcacagt | 5700 |
| ggtagttaga ggtgaaaagt agagctgtca agcccaaggg cttagcttta gggctcctcc | 5760 |
| tgagttcggc ccacagtaga agcaagattt taactagccc cttttcctct tcaccctccc | 5820 |
| atgatgcgca gtgttcagaa agctggtaag tcctagggat ttccagaagt agcctgcaga | 5880 |
| agaaggtaag tttgaaagcc actccagggg tcctgatgct gtcatgctca gtgagccatt | 5940 |
| ttacagttct ccaaagtcta gccctgtttc ggacctgcac ttcacctcta agttatgtac | 6000 |
| aactcaacct gcatccctct aaaagtccta tatccatatt caccattggc taatttgagg | 6060 |
| ccctgagtgg gccttgaatg ctaaaaagaa gcagggtacg cagggctaca tgtagatacc | 6120 |
| acaccaaggc tggaggctgg tctgtcataa gacagaaaga aagacgctgg gcccaatttt | 6180 |
| gacttggcca ggggacacct tggtgtgttt gttatcttta tctgtgggta ggctagctga | 6240 |
| cccatctcct tgagtcattc cctttgggaa accccactgc cagtattgat ctcctttttg | 6300 |
| ccttgtactg aatgacacat tacctccaca ctctcccgga ctaggtggtc aacagggcca | 6360 |
| cagggttgct ttctgtcttt ggtggggcag gggagttgac agggatgagg gtccaaggaa | 6420 |
| taagcatgaa tgacaagaaa acaagggaaa gagttaacct gtcacatagc aggttaactt | 6480 |
| tttcagggtt tgcagttaga ggtattcgac cattcactgg ctgagccaga tcacgggaac | 6540 |
| ttgagagctt ttactgtgat tcttcaatgt aaaaaataaa caacaatgtc aaactgtgtt | 6600 |
| tatatgattt gtataaagcc tttttaagat tactatttaa ataaacatta taccagaga | 6659 |

SEQ ID NO: 4
Met Asp Met Phe Pro Leu Thr Trp Val Phe Leu Ala Leu Tyr Phe Ser
Arg His Gln Val Arg Gly Gln Pro Asp Pro Pro Cys Gly Gly Arg Leu
Asn Ser Lys Asp Ala Gly Tyr Ile Thr Ser Pro Gly Tyr Pro Gln Asp
Tyr Pro Ser His Gln Asn Cys Glu Trp Ile Val Tyr Ala Pro Glu Pro
Asn Gln Lys Ile Val Leu Asn Phe Asn Pro His Phe Glu Ile Glu Lys
His Asp Cys Lys Tyr Asp Phe Ile Glu Ile Arg Asp Gly Asp Ser Glu
Ser Ala Asp Leu Leu Gly Lys His Cys Gly Asn Ile Ala Pro Pro Thr
Ile Ile Ser Ser Gly Ser Met Leu Tyr Ile Lys Phe Thr Ser Asp Tyr
Ala Arg Gln Gly Ala Gly Phe Ser Leu Arg Tyr Glu Ile Phe Lys Thr
Gly Ser Glu Asp Cys Ser Lys Asn Phe Thr Ser Pro Asn Gly Thr Ile
Glu Ser Pro Gly Phe Pro Glu Lys Tyr Pro His Asn Leu Asp Cys Thr
Phe Thr Ile Leu Ala Lys Pro Lys Met Glu Ile Ile Leu Gln Phe Leu
Ile Phe Asp Leu Glu His Asp Pro Leu Gln Val Gly Glu Gly Asp Cys
Lys Tyr Asp Trp Leu Asp Ile Trp Asp Gly Ile Pro His Val Gly Pro
Leu Ile Gly Lys Tyr Cys Gly Thr Lys Thr Pro Ser Glu Leu Arg Ser
Ser Thr Gly Ile Leu Ser Leu Thr Phe His Thr Asp Met Ala Val Ala
Lys Asp Gly Phe Ser Ala Arg Tyr Tyr Leu Val His Gln Glu Pro Leu
Glu Asn Phe Gln Cys Asn Val Pro Leu Gly Met Glu Ser Gly Arg Ile
Ala Asn Glu Gln Ile Ser Ala Ser Ser Thr Tyr Ser Asp Gly Arg Trp
Thr Pro Gln Gln Ser Arg Leu His Gly Asp Asp Asn Gly Trp Thr Pro
Asn Leu Asp Ser Asn Lys Glu Tyr Leu Gln Val Asp Leu Arg Phe Leu
Thr Met Leu Thr Ala Ile Ala Thr Gln Gly Ala Ile Ser Arg Glu Thr
Gln Asn Gly Tyr Tyr Val Lys Ser Tyr Lys Leu Glu Val Ser Thr Asn
Gly Glu Asp Trp Met Val Tyr Arg His Gly Lys Asn His Lys Val Phe
Gln Ala Asn Asn Asp Ala Thr Glu Val Val Leu Asn Lys Leu His Ala
Pro Leu Leu Thr Arg Phe Val Arg Ile Arg Pro Gln Thr Trp His Ser
Gly Ile Ala Leu Arg Leu Glu Leu Phe Gly Cys Arg Val Thr Asp Ala
Pro Cys Ser Asn Met Leu Gly Met Leu Ser Gly Leu Ile Ala Asp Ser
Gln Ile Ser Ala Ser Ser Thr Gln Glu Tyr Leu Trp Ser Pro Ser Ala
Ala Arg Leu Val Ser Ser Arg Ser Gly Trp Phe Pro Arg Ile Pro Gln
Ala Gln Pro Gly Glu Glu Trp Leu Gln Val Asp Leu Gly Thr Pro Lys
Thr Val Lys Gly Val Ile Ile Gln Gly Ala Arg Gly Gly Asp Ser Ile
Thr Ala Val Glu Ala Arg Ala Phe Val Arg Lys Phe Lys Val Ser Tyr
Ser Leu Asn Gly Lys Asp Trp Glu Tyr Ile Gln Asp Pro Arg Thr Gln
Gln Pro Lys Leu Phe Glu Gly Asn Met His Tyr Asp Thr Pro Asp Ile
Arg Arg Phe Asp Pro Ile Pro Ala Gln Tyr Val Arg Val Tyr Pro Glu
Arg Trp Ser Pro Ala Gly Ile Gly Met Arg Leu Glu Val Leu Gly Cys
Asp Trp Thr Asp Ser Lys Pro Thr Val Glu Thr Leu Gly Pro Thr Val
Lys Ser Glu Glu Thr Thr Thr Pro Tyr Pro Thr Glu Glu Glu Ala Thr
Glu Cys Gly Glu Asn Cys Ser Phe Glu Asp Asp Lys Asp Leu Gln Leu
Pro Ser Gly Phe Asn Cys Asn Phe Asp Phe Leu Glu Glu Pro Cys Gly
Trp Met Tyr Asp His Ala Lys Trp Leu Arg Thr Thr Trp Ala Ser Ser
Ser Ser Pro Asn Asp Arg Thr Phe Pro Asp Asp Arg Asn Phe Leu Arg
Leu Gln Ser Asp Ser Gln Arg Glu Gly Gln Tyr Ala Arg Leu Ile Ser
Pro Pro Val His Leu Pro Arg Ser Pro Val Cys Met Glu Phe Gln Tyr
Gln Ala Thr Gly Gly Arg Gly Val Ala Leu Gln Val Val Arg Glu Ala
Ser Gln Glu Ser Lys Leu Leu Trp Val Ile Arg Glu Asp Gln Gly Gly
Glu Trp Lys His Gly Arg Ile Ile Leu Pro Ser Tyr Asp Met Glu Tyr
Gln Ile Val Phe Glu Gly Val Ile Gly Lys Gly Arg Ser Gly Glu Ile
Ala Ile Asp Asp Ile Arg Ile Ser Thr Asp Val Pro Leu Glu Asn Cys
Met Glu Pro Ile Ser Ala Phe Ala Gly Glu Asn Phe Lys Val Asp Ile
Pro Glu Ile His Glu Arg Glu Gly Tyr Glu Asp Glu Ile Asp Asp Glu
Tyr Glu Val Asp Trp Ser Asn Ser Ser Ser Ala Thr Ser Gly Ser Gly
Ala Pro Ser Thr Asp Lys Glu Lys Ser Trp Leu Tyr Thr Leu Asp Pro
Ile Leu Ile Thr Ile Ile Ala Met Ser Ser Leu Gly Val Leu Leu Gly
Ala Thr Cys Ala Gly Leu Leu Leu Tyr Cys Thr Cys Ser Tyr Ser Gly
Leu Ser Ser Arg Ser Cys Thr Thr Leu Glu Asn Tyr Asn Phe Glu Leu
Tyr Asp Gly Leu Lys His Lys Val Lys Met Asn His Gln Lys Cys Cys
Ser Glu Ala

What is claimed herein:

1. A semaphorin (SEMA) polypeptide, wherein the proproteinase cleavage motif is inactivated by mutation, and wherein the proproteinase cleavage motif is selected from the group consisting of: RXXR, RXKR, RXRR, SEQ ID NO: 15, and SEQ ID NO: 16.

2. The SEMA polypeptide of claim 1, wherein the proproteinase cleavage motif is selected from the group consisting of: SEQ ID NO: 14, SEQ ID NO: 8, SEQ ID NO: 13, SEQ ID NO: 11, SEQ ID NO: 12, and SEQ ID NO: 10.

3. The SEMA polypeptide of claim 1, wherein its wild-type SEMA polypeptide binds to neuropilin 2, and wherein the mutant SEMA polypeptide retains the ability to bind to neuropilin 2.

4. The SEMA polypeptide of claim 1, wherein the SEMA polypeptide is a human SEMA polypeptide.

5. The SEMA polypeptide of claim 1, wherein the SEMA polypeptide is selected from a SEMA 3A polypeptide, SEMA 3C polypeptide, a SEMA 3F polypeptide and a SEMA 3G polypeptide.

6. The SEMA polypeptide of claim 2, wherein the proproteinase cleavage motif comprises amino acids:
(i) 582-586 of SEQ ID NO: 1,
(ii) 583-586 of SEQ ID NO: 1,
(iii) 550-555 of SEQ ID NO: 5,
(iv) 551-555 of SEQ ID NO: 5,
(v) 548-552 of SEQ ID NO: 6,
(vi) 549-552 of SEQ ID NO: 6,
(vii) 557-561 of SEQ ID NO: 7, or
(viii) 558-561 of SEQ ID NO:7.

7. The SEMA polypeptide of claim 2, wherein the proproteinase cleavage motif of SEQ ID NO: 8 is inactivated by mutating the second and fourth arginines in the motif.

8. The SEMA polypeptide of claim 2, wherein the proproteinase cleavage motif of SEQ ID NO: 8 is inactivated by mutating arginine 583 and arginine 586 of SEQ ID NO: 1 to alanine.

9. A nucleic acid molecule encoding any one of the SEMA polypeptides of claim 1.

10. The nucleic acid molecule of claim 9, wherein the nucleic acid molecule is a cDNA or a modified RNA.

11. A vector comprising the nucleic acid molecule of claim 9.

12. The vector of claim 11, wherein the vector is a viral vector.

13. A cell comprising the nucleic acid of claim 9.

14. A pharmaceutical composition comprising the SEMA polypeptide claim 1.

15. A method of inhibiting transplant or allograft rejection in a subject in need thereof, the method comprising: administering to the subject a therapeutically effective amount of a pharmaceutical composition of claim 14.

16. A method of inhibiting transplant or allograft rejection in a subject, the method comprising: contacting transplant tissue with an amount of a pharmaceutical composition of claim 14 that is effective to suppress the immune system of the subject.

17. A method of suppressing the immune system in a subject in need thereof comprising administering to the subject a pharmaceutical composition of claim 14.

18. A method of treating an inflammatory condition in a subject in need thereof comprising administering to the subject a pharmaceutical composition of claim 14.

19. The method of claim 18, wherein the inflammatory condition is an autoimmune disease.

20. A multispecific agent comprising a semaphorin polypeptide that binds to neuropilin 2, and an agent that binds an immunomodulator polypeptide.


SEQUENCE LISTING (16 sequences)

SEQ ID NO: 1 - protein, 785 aa, Homo sapiens
MLVAGLLLWASLLTGAWPSFPTQDHLPATPRVRLSFKELKATGTAHFFNFLLNTTDYRILLKDEDHDRMYVGSKDYVLSLDLHDINREPLIIHWAASPQRIEECVLSGKDVNGECGNFVRLIQPWNRTHLYVCGTGAYNPMCTYVNRGRRAQATPWTQTQAVRGRGSRATDGALRPMPTAPRQDYIFYLEPERLESGKGKCPYDPKLDTASALINEELYAGVYIDFMGTDAAIFRTLGKQTAMRTDQYNSRWLNDPSFIHAELIPDSAERNDDKLYFFFRERSAEAPQSPAVYARIGRICLNDDGGHCCLVNKWSTFLKARLVCSVPGEDGIETHFDELQDVFVQQTQDVRNPVIYAVFTSSGSVFRGSAVCVYSMADIRMVFNGPFAHKEGPNYQWMPFSGKMPYPRPGTCPGGTFTPSMKSTKDYPDEVINFMRSHPLMYQAVYPLQRRPLVVRTGAPYRLTTIAVDQVDAADGRYEVLFLGTDRGTVQKVIVLPKDDQELEELMLEEVEVFKDPAPVKTMTISSKRQQLYVASAVGVTHLSLHRCQAYGAACADCCLARDPYCAWDGQACSRYTASSKRRSRRQDVRHGNPIRQCRGFNSNANKNAVESVQYGVAGSAAFLECQPRSPQATVKWLFQRDPGDRRREIRAEDRFLRTEQGLLLRALQLSDRGLYSCTATENNFKHVVTRVQLHVLGRDAVHAALFPPLSMSAPPPPGAGPPTPPYQELAQLLAQPEVGLIHQYCQGYWRHVPPSPREAPGAPRSPEPQDQKKPRNRRHHPPDT

SEQ ID NO: 2 - DNA, 3607 bp, Homo sapiens
  source 1..3607: mol_type = unassigned DNA
gagcgcccctgagccttcccatggcccgggctggggcccgggccctcggctgctgacgcgcccgaagcccgcggaaccggttaagccgcggccgcggcgccgatcccggctgaggcgcagcggcgagaggtcgcgggcagggccatggccccggggggccgctagcgcggaccggcccaacgggagccgctccgtgccgccgccgccgcccgggcgcccaggccccgccgctgcggaagaggtttctagagagtggagcctgcttcctgggccctaggcccctcccacaatgcttgtcgccggtcttcttctctgggcttccctactgaccggggcctggccatccttccccacccaggaccacctcccggccacgccccgggtccggctctcattcaaagagctgaaggccacaggcaccgcccacttcttcaacttcctgctcaacacaaccgactaccgaatcttgctcaaggacgaggaccacgaccgcatgtacgtgggcagcaaggactacgtgctgtccctggacctgcacgacatcaaccgcgagcccctcattatacactgggcagcctccccacagcgcatcgaggaatgcgtgctctcaggcaaggatgtcaacggcgagtgtgggaacttcgtcaggctcatccagccctggaaccgaacacacctgtatgtgtgcgggacaggtgcctacaaccccatgtgcacctatgtgaaccgcggacgccgcgcccaggccacaccatggacccagactcaggcggtcagaggccgcggcagcagagccacggatggtgccctccgcccgatgcccacagccccacgccaggattacatcttctacctggagcctgagcgactcgagtcagggaagggcaagtgtccgtacgatcccaagctggacacagcatcggccctcatcaatgaggagctctatgctggtgtgtacatcgattttatgggcactgatgcagccatcttccgcacacttggaaagcagacagccatgcgcacggatcagtacaactcccggtggctgaacgacccgtcgttcatccatgctgagctcattcctgacagtgcggagcgcaatgatgataagctttacttcttcttccgtgagcggtcggcagaggcgccgcagagccccgcggtgtacgcccgcatcgggcgcatttgcctgaacgatgacggtggtcactgttgcctggtcaacaagtggagcacattcctgaaggcgcggctcgtctgctctgtcccgggcgaggatggcattgagactcactttgatgagctccaggacgtgtttgtccagcagacccaggacgtgaggaaccctgtcatttacgctgtctttacctcctctggctccgtgttccgaggctctgccgtgtgtgtctactccatggctgatattcgcatggtcttcaacgggccctttgcccacaaagaggggcccaactaccagtggatgcccttctcagggaagatgccctacccacggccgggcacgtgccctggtggaaccttcacgccatctatgaagtccaccaaggattatcctgatgaggtgatcaacttcatgcgcagccacccactcatgtaccaggccgtgtaccctctgcagcggcggcccctggtagtccgcacaggtgctccctaccgccttaccactattgccgtggaccaggtggatgcagccgacgggcgctatgaggtgcttttcctgggcacagaccgcgggacagtgcagaaggtcattgtgctgcccaaggatgaccaggagttggaggagctcatgctggaggaggtggaggtcttcaaggatccagcacccgtcaagaccatgaccatctcttctaagaggcaacaactctacgtggcgtcagccgtgggtgtcacacacctgagcctgcaccgctgccaggcgtatggggctgcctgtgctgactgctgccttgcccgggacccttactgtgcctgggatggccaggcctgctcccgctatacagcatcctccaagaggcggagccgccggcaggacgtccggcacggaaaccccatcaggcagtgccgtgggttcaactccaatgccaacaagaatgccgtggagtctgtgcagtatggcgtggccggcagcgcagccttccttgagtgccagccccgctcgccccaagccactgttaagtggctgttccagcgagatcctggtgaccggcgccgagagattcgtgcagaggaccgcttcctgcgcacagagcagggcttgttgctccgtgcactgcagctcagcgatcgtggcctctactcctgcacagccactgagaacaactttaagcacgtcgtcacacgagtgcagctgcatgtactgggccgggacgccgtccatgctgccctcttcccaccactgtccatgagcgccccgccacccccaggcgcaggccccccaacgcctccttaccaggagttagcccagctgctggcccagccagaagtgggcctcatccaccagtactgccagggttactggcgccatgtgccccccagccccagggaggctccaggggcaccccggtctcctgagccccaggaccagaaaaagccccggaaccgccggcaccaccctccggacacatgaggccagctgcctgtgcctgccatgggccagcctagcccttgtcccttttaatataaaagatatatatatatatatatatatataaaatatctatattctatacacaccctgcccctgcaaagacagtatttattggtgggttgaatatagcctgcctcagtggcagcatcctccaaaacttagacccatgctggtcagagacggcagaaaacagagcctgcctaaccaggcccagccagttggtggggccaggccaggaccacacagtccccagactcagctggaagtctacctgctggacagcctccgccaagatctacaggacaaagggagggagcaagccctactcggatggggcacggactgtccaccttttctgatgtgtgttgtcagcctgtgctgtggcatagacatggatgcgaggaccactttggagactggggtggcctcaagagcacacagagaagggaagaaggggccatcacaggatgccagcccctgcctgggttgggggcactcagccacgaccagccccttcctgggtatttattctctatttattggggataggagaagaggcatcctgcctgggtgggacagcctcttcagccccttctcccctccccgcctggccagggcagggccaccccactctacctccttagctttccctgtgccactttgactcagaggctgggagcatagcagaggggccaggcccaggcagagctgacgggaggccccagctctgaggggagggggtccgtggtagaggcctggggccggtagaggctccccagggctcccttatgtccaccacttcaggggatgggtgtggatgtaattagctctggggggcagttgggtagatgggtgggggctcctggtggccttctgctgcccaggccacagccgcctttgggttccatcttgctaataaacactggctctgggactaga

SEQ ID NO: 3 - DNA, 6659 bp, Homo sapiens
  source 1..6659: mol_type = unassigned DNA
agagatcgcgagcgaggcaccagcctgcagccggcccccagcacatcctcagccgcacagacactcggcgaggtggaggtgagggcgggcgccagcgaactcggagaggggctcgctcactcccaggcgatcccagccgccaccgccgccgcaccagcagcagcaacagcagcagcagcttccttcctcagactcccctcgagaggctggccaagcgggtgtagccgttgggggaggctcccgccgggggaacccggcgaggacaagagcagggcggccgccttccactcgggctgtccggcggcggctgcctccgcccgtgtgtccgtcaagggtgccgcgggatgtgtgtcagtttacgcctctgagatcacacagctgcctgggggccgtgtgatgcccaaggcaagtcttggttttaattattattattatcattattgttacgcttggctttcgggaaatactcgtgatatttgtaggataaaggaaatgacactttgaggaactggagagaacatatatgcgttttgtttttaagaggaaaaccgtgttctcttcccggcttgttccctctttgctgatttcaggagctactctcctcctggtgaggtggaaattccagcaagaatagaggtgaagacaagccaccaggactcaggagggaaacgctgaccattagaaacctctgcataagacgttgtaaggaggaaaataaaagagagaaaaacacaaagatttaaacaagaaacctacgaacccagctctggaaagagccaccttctccaaaatggatatgtttcctctcacctgggttttcttagccctctacttttcaagacaccaagtgagaggccaaccagacccaccgtgcggaggtcgtttgaattccaaagatgctggctatatcacctctcccggttacccccaggactacccctcccaccagaactgcgagtggattgtttacgcccccgaacccaaccagaagattgtcctcaacttcaaccctcactttgaaatcgagaagcacgactgcaagtatgactttatcgagattcgggatggggacagtgaatccgcagacctcctgggcaaacactgtgggaacatcgccccgcccaccatcatctcctcgggctccatgctctacatcaagttcacctccgactacgcccggcagggggcaggcttctctctgcgctacgagatcttcaagacaggctctgaagattgctcaaaaaacttcacaagccccaacgggaccatcgaatctcctgggtttcctgagaagtatccacacaacttggactgcacctttaccatcctggccaaacccaagatggagatcatcctgcagttcctgatctttgacctggagcatgaccctttgcaggtgggagagggggactgcaagtacgattggctggacatctgggatggcattccacatgttggccccctgattggcaagtactgtgggaccaaaacaccctctgaacttcgttcatcgacggggatcctctccctgacctttcacacggacatggcggtggccaaggatggcttctctgcgcgttactacctggtccaccaagagccactagagaactttcagtgcaatgttcctctgggcatggagtctggccggattgctaatgaacagatcagtgcctcatctacctactctgatgggaggtggacccctcaacaaagccggctccatggtgatgacaatggctggacccccaacttggattccaacaaggagtatctccaggtggacctgcgctttttaaccatgctcacggccatcgcaacacagggagcgatttccagggaaacacagaatggctactatgtcaaatcctacaagctggaagtcagcactaatggagaggactggatggtgtaccggcatggcaaaaaccacaaggtatttcaagccaacaacgatgcaactgaggtggttctgaacaagctccacgctccactgctgacaaggtttgttagaatccgccctcagacctggcactcaggtatcgccctccggctggagctcttcggctgccgggtcacagatgctccctgctccaacatgctggggatgctctcaggcctcattgcagactcccagatctccgcctcttccacccaggaatacctctggagccccagtgcagcccgcctggttagcagccgctcgggctggttccctcgaatccctcaggcccagcccggtgaggagtggcttcaggtagatctgggaacacccaagacagtgaaaggtgtcatcatccagggagcccgcggaggagacagtatcactgctgtggaagccagagcatttgtgcgcaagttcaaagtctcctacagcctaaacggcaaggactgggaatacattcaggaccccaggacccagcagccaaagctgttcgaagggaacatgcactatgacacccctgacatccgaaggtttgaccccattccggcacagtatgtgcgggtatacccggagaggtggtcgccggcggggattgggatgcggctggaggtgctgggctgtgactggacagactccaagcccacggtagagacgctgggacccactgtgaagagcgaagagacaaccaccccctaccccaccgaagaggaggccacagagtgtggggagaactgcagctttgaggatgacaaagatttgcagctcccttcgggattcaattgcaacttcgatttcctcgaggagccctgtggttggatgtatgaccatgccaagtggctccggaccacctgggccagcagctccagcccaaacgaccggacgtttccagatgacaggaatttcttgcggctgcagagtgacagccagagagagggccagtatgcccggctcatcagcccccctgtccacctgccccgaagcccggtgtgcatggagttccagtaccaggccacgggcggccgcggggtggcgctgcaggtggtgcgggaagccagccaggagagcaagttgctgtgggtcatccgtgaggaccagggcggcgagtggaagcacgggcggatcatcctgcccagctacgacatggagtaccagattgtgttcgagggagtgatagggaaaggacgttccggagagattgccattgatgacattcggataagcactgatgtcccactggagaactgcatggaacccatctcggcttttgcaggtgagaattttaaagtggacatcccagaaatacatgagagagaaggatatgaagatgaaattgatgatgaatacgaggtggactggagcaattcttcttctgcaacctcagggtctggcgccccctcgaccgacaaagaaaagagctggctgtacaccctggatcccatcctcatcaccatcatcgccatgagctcactgggcgtcctcctgggggccacctgtgcaggcctcctgctctactgcacctgttcctactcgggcctgagctcccgaagctgcaccacactggagaactacaacttcgagctctacgatggccttaagcacaaggtcaagatgaaccaccaaaagtgctgctccgaggcatgacggattgcacctgaatcctatctgacgtttcattccagcaagaggggctggggaagattacatttttttttcctttggaaactgaatgccataatctcgatcaaaccgatccagaataccgaaggtatggacaggacagaaaagcgagtcgcaggaggaagggagatgcagccgcacaggggatgattaccctcctaggaccgcggtggctaagtcattgcaggaacggggctgtgttctctgctgggacaaaacaggagctcatctctttggggtcacagttctattttgtttgtgagtttgtattattattattattattattattattatattttatttctttggtctgtgagcaactcaaagaggcagaagaggagaatgacttttccagaatagaagtggagcagtgatcattattctccgctttctctttctaatcaacacttgaaaagcaaagtgtcttttcagcctttccatctttacaaataaaactcaaaaaagccgtccagcttatcccatcctctgattgtcttctgacttaagggatttactgtggtgtaggttctgccagccaaccctacaagctgccatttccagtcctagcatttaagtaggatgttgttgcctttaacttttcttatccaggggaaaattgccattttagggtcagcatgaacagctctttcttgtatgcgatttaaaacaaactggaaaggaaacttcacacgtcaaaatccatagaagcgcctggacgaggcttaaagtgctttgtgagtgaataggagccattcgctaattctagacccacagtgtctggtggtggggcttcccttgtggggcttctggtggtggttttgccttttcttttccctcctccatgttcttctaaaacatatacatatatacatacacacatacacatattcttcaggtctctaagcccctggaagcagcattgtgtgatattctcagaggcaggggaaaatagagggaaaaatagagactattggtatgttctccccatcagcgagttattgtaactggtcaccactggacgggaaggagaacagaggagagggaaagagaagcccaacctctgtgatcatatgagggccaaggctgagcagtgtagacagagaccctttgaaatgcatttgtctctcaaatagactagtaaacaccgacttctcctttgggttacaaacaccatttcaacctttcgggagagtcagagctaggatgtacaagaactgattctaaccagaagtccgcaagtactgtggacaagaatgcttaaccatgctgcttcagccttgagagacctaggttcttacacatatgcacacacgcatacacacatgcacgcacacacacatacacacatgcacgcacgcacgcatgcacaccaatttatgtttttattaagtgccttgaaaaaatgaagaaaaatgtattttccctttatgtaaaaattagtgaatatcttatgaattaaggcattcctctttccctaaccccgatggctccattcccaagtaccccaactcactgctgatcctattaaaggaatgagtcctgctacccgagtggtagtcatagccctagatgactctcaactactcttcaaagggaggcatcaggaatagaatgaaactgtgtgaaggataagattgttcgcatcaagatccaaatcttgatttcatattaacgcctaaggattgcctgtgtgctggaaatatatttgaaactcaaccagtatgcccagcctattgcatatcattgtcagaccatttttgctgctgtggtcacccacgatttcatttgtcttatacccaggtgaaaggggaagggtgaatgggactggctggttcctttaaatgttaacttatggaaatgctagttcaaatggtaatgtcacagtgttttgtatgcagagagcaagagttcaaccaacagctgtttattcatgtgtgtgtgtctttgctgctttgagttctctgtatctactgtgtatgtgaatggtcatgtgggactcagtggtggtgttgtgactttgacctagggtccgagtgtcacagctgatcttggcactcggcactcattggcacagtggtagttagaggtgaaaagtagagctgtcaagcccaagggcttagctttagggctcctcctgagttcggcccacagtagaagcaagattttaactagccccttttcctcttcaccctcccatgatgcgcagtgttcagaaagctggtaagtcctagggatttccagaagtagcctgcagaagaaggtaagtttgaaagccactccaggggtcctgatgctgtcatgctcagtgagccattttacagttctccaaagtctagccctgtttcggacctgcacttcacctctaagttatgtacaactcaacctgcatccctctaaaagtcctatatccatattcaccattggctaatttgaggccctgagtgggccttgaatgctaaaaagaagcagggtacgcagggctacatgtagataccacaccaaggctggaggctggtctgtcataagacagaaagaaagacgctgggcccaattttgacttggccaggggacaccttggtgtgtttgttatctttatctgtgggtaggctagctgacccatctccttgagtcattccctttgggaaaccccactgccagtattgatctcctttttgccttgtactgaatgacacattacctccacactctcccggactaggtggtcaacagggccacagggttgctttctgtctttggtggggcaggggagttgacagggatgagggtccaaggaataagcatgaatgacaagaaaacaagggaaagagttaacctgtcacatagcaggttaactttttcagggtttgcagttagaggtattcgaccattcactggctgagccagatcacgggaacttgagagcttttactgtgattcttcaatgtaaaaaataaacaacaatgtcaaactgtgtttatatgatttgtataaagcctttttaagattactatttaaataaacattataccagaga

SEQ ID NO: 4 - protein, 931 aa, Homo sapiens
MDMFPLTWVFLALYFSRHQVRGQPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAGENFKVDIPEIHEREGYEDEIDDEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 5 - protein, 771 aa, Homo sapiens [sequence omitted]

SEQ ID NO: 6 - protein, 751 aa, Homo sapiens
MAFRTICVLVGVFICSICVKGSSQPQARVYLTFDELRETKTSEYFSLSHHPLDYRILLMDEDQDRIYVGSKDHILSLNINNISQEALSVFWPASTIKVEECKMAGKDPTHGCGNFVRVIQTFNRTHLYVCGSGAFSPVCTYLNRGRRSEDQVFMIDSKCESGKGRCSFNPNVNTVSVMINEELFSGMYIDFMGTDAAIFRSLTKRNAVRTDQHNSKWLSEPMFVDAHVIPDGTDPNDAKVYFFFKEKLTDNNRSTKQIHSMIARICPNDTGGLRSLVNKWTTFLKARLVCSVTDEDGPETHFDELEDVFLLETDNPRTTLVYGIFTTSSSVFKGSAVCVYHLSDIQTVFNGPFAHKEGPNHQLISYQGRIPYPRPGTCPGGAFTPNMRTTKEFPDDVVTFIRNHPLMYNSIYPIHKRPLIVRIGTDYKYTKIAVDRVNAADGRYHVLFLGTDRGTVQKVVVLPTNNSVSGELILEELEVFKNHAPITTMKISSKKQQLYVSSNEGVSQVSLHRCHIYGTACADCCLARDPYCAWDGHSCSRFYPTGKRRSRRQDVRHGNPLTQCRGFNLKAYRNAAEIVQYGVKNNTTFLECAPKSPQASIKWLLQKDKDRRKEVKLNERIIATSQGLLIRSVQGSDQGLYHCIATENSFKQTIAKINFKVLDSEMVAVVTDKWSPWTWASSVRALPFHPKDIMGAFSHSEMQMINQYCKDTRQQHQQGDESQKMRGDYGKLKALINSRKSRNRRNQLPES

SEQ ID NO: 7 - protein, 782 aa, Homo sapiens
MAPSAWAICWLLGGLLLHGGSSGPSPGPSVPRLRLSYRDLLSANRSAIFLGPQGSLNLQAMYLDEYRDRLFLGGLDALYSLRLDQAWPDPREVLWPPQPGQREECVRKGRDPLTECANFVRVLQPHNRTHLLACGTGAFQPTCALITVGHRGEHVLHLEPGSVESGRGRCPHEPSRPFASTFIDGELYTGLTADFLGREAMIFRSGGPRPALRSDSDQSLLHDPRFVMAARIPENSDQDNDKVYFFFSETVPSPDGGSNHVTVSRVGRVCVNDAGGQRVLVNKWSTFLKARLVCSVPGPGGAETHFDQLEDVFLLWPKAGKSLEVYALFSTVSAVFQGFAVCVYHMADIWEVFNGPFAHRDGPQHQWGPYGGKVPFPRPGVCPSKMTAQPGRPFGSTKDYPDEVLQFARAHPLMFWPVRPRHGRPVLVKTHLAQQLHQIVVDRVEAEDGTYDVIFLGTDSGSVLKVIALQAGGSAEPEEVVLEELQVFKVPTPITEMEISVKRQMLYVGSRLGVAQLRLHQCETYGTACAECCLARDPYCAWDGASCTHYRPSLGKRRFRRQDIRHGNPALQCLGQSQEEEAVGLVAATMVYGTEHNSTFLECLPKSPQAAVRWLLQRPGDEGPDQVKTDERVLHTERGLLFRRLSRFDAGTYTCTTLEHGFSQTVVRLALVVIVASQLDNLFPPEPKPEEPPARGGLASTPPKAWYKDILQLIGFANLPRVDEYCERVWCRGTTECSGCFRSRSRGKQARGKSWAGLELGKKMKSRVHAEHNRTPREVEAT

SEQ ID NO: 8 - protein, 5 aa, Homo sapiens
RRSRR

SEQ ID NO: 9 - protein, 5 aa, synthetic construct
  REGION 1..5: note = Description of Artificial Sequence: Synthetic peptide
RASRA

SEQ ID NO: 10 - protein, 5 aa, synthetic construct
  REGION 1..5: note = Description of Artificial Sequence: Synthetic peptide
RRFRR

SEQ ID NO: 11 - protein, 5 aa, synthetic construct
  REGION 1..5: note = Description of Artificial Sequence: Synthetic peptide
RRTRR

SEQ ID NO: 12 - protein, 4 aa, synthetic construct
  REGION 1..4: note = Description of Artificial Sequence: Synthetic peptide
RFRR

SEQ ID NO: 13 - protein, 4 aa, synthetic construct
  REGION 1..4: note = Description of Artificial Sequence: Synthetic peptide
RTRR

SEQ ID NO: 14 - protein, 4 aa, synthetic construct
  REGION 1..4: note = Description of Artificial Sequence: Synthetic peptide
RSRR

SEQ ID NO: 15 - protein, 5 aa, synthetic construct
  REGION 1..5: note = Description of Artificial Sequence: Synthetic peptide
  VARIANT 3: note = MOD_RES - Any amino acid
RRXRR

SEQ ID NO: 16 - protein, 5 aa, synthetic construct
  REGION 1..5: note = Description of Artificial Sequence: Synthetic peptide
  VARIANT 3: note = MOD_RES - Any amino acid
RRXKR